(19) United States
(12) Patent Application Publication
(10) Pub. No.: US 2025/0067643 A1
(43) Pub. Date: Feb. 27, 2025

(54) Gas-Liquid Falling Film Equilibration System and Method Of Use

(71) Applicant: Smithsonian Institution, Washington, DC (US)

(72) Inventor: Alexander Whitman MILLER, Annapolis, MD (US)

(21) Appl. No.: 18/799,690

(22) Filed: Aug. 9, 2024

Related U.S. Application Data
(63) Continuation of application No. PCT/US2023/012958, filed on Feb. 13, 2023.
(60) Provisional application No. 63/309,334, filed on Feb. 11, 2022.

(51) Int. Cl. G01N 1/38; G01N 1/40; G01N 33/18

(52) U.S. Cl. CPC G01N 1/38; G01N 1/4022; G01N 33/18

(57) ABSTRACT

The current disclosure provides a gas-liquid falling film equilibration apparatus that includes one or more temperature control mechanisms, systems incorporating the apparatus, and methods of their use. The apparatus finds use in making continuous and discrete measurements of one or more dissolved gases and/or volatile materials in a variety of liquids, including the measurement of carbon dioxide and methane in water.

### CROSS-REFERENCE TO RELATED APPLICATIONS

This application claims the benefit of U.S. Provisional Application No. 63/309,334, filed Feb. 11, 2022.

### TECHNICAL FIELD

This disclosure is directed to the measurement of industrially and/or environmentally important gases or volatile materials (e.g., CO₂ or methane) in liquids and gas/falling-liquid film equilibrator apparatus for such measurements. The equilibrators permit separation of some or all of the gases and/or volatile substances in liquids into a gas phase in which the gases and/or volatiles of interest can be measured. Such measurements can be complicated by the presence of the liquid, contaminants in the liquid, and fouling (e.g., biofouling) of the apparatus used for measurements and difficulties with accuracy and precision.

### BACKGROUND

The measurement/monitoring of gases in various liquids is of both environmental and industrial importance. Various systems for the measurement of gases, including but not limited to CO₂ and methane, have been devised. Included in those systems are apparatus that assess the concentration of gases in the liquid directly (e.g., by spectral analysis or chemical reaction in the liquid phase) and apparatus that force the gas from the solution by physical/chemical means (e.g., addition of acid, elevation of temperature, etc.) thereby permitting measurement in the gas phase.

Measurement of gases including greenhouse gases such as carbon dioxide (CO₂) and methane is of increasing importance as those gases have an effect on the regulation of the earth's temperature. It is estimated that roughly 30% of anthropogenic CO₂ leaves the atmosphere and enters the earth's oceans and other large bodies of water. These water bodies typically act as large sinks of CO₂, wherein dissolved CO₂ becomes carbonic acid, carbonate, and bicarbonate, with concomitant changes in pH. Unfortunately, devices for directly measuring pH in the natural environment are unreliable when deployed for any length of time, especially in systems with high productivity and/or sediment loads. In coastal systems, such as estuaries, where changes in salinity are common and biofouling extensive, measuring pH can be burdensome and inaccurate. Alternatively, measurements of changes in the partial pressure of CO₂ in the ocean can provide valuable and reliable information about changes in the acidity of the ocean. Nearshore coastal water pH measurements can also be made providing similar information.

Methods for measuring pCO₂ in oceans have mainly focused on measuring acidification in open ocean settings. These methods assume that acidification is driven by a stable air-sea CO₂ equilibrium, such that measurement of the ocean's pCO₂ is reflective of atmospheric pCO₂. The technology depends on large, expensive, and sparse autonomous buoys to characterize hundreds to thousands of km² of ocean surrounding them. Buoy data are supplemented by data taken during ocean transits by large, expensive, and sparse oceanographic research vessels.

Due to the complex make-up of nearshore coastal waters, an air-sea equilibrium rarely occurs, and measurements must be made at a higher frequency over space and time. Increased frequencies can assist to reliably characterize pCO₂ and pH. In nearshore waters the carbon cycle is much more complicated than in the open ocean, and land-sea interactions and ecosystem metabolism are frequently more acute drivers of pCO₂ than air-sea interactions. Nearshore waters are further complicated by biological activities such as photosynthesis and respiration and the pCO₂ of the water is far more dynamic than in the open ocean. Changes in pCO₂ are more rapid than in open ocean waters and pCO₂ can vary significantly over very short distances and time spans. Measurements must therefore be made much more frequently and much more densely in order to capture the natural temporal and spatial variability present.

Challenging environmental conditions also adversely affect the accurate measurement and long-term monitoring of gases (e.g., CO₂) and other volatile organic and inorganic substances that dissolve in water (e.g., radon, methane, etc.) or other liquids subject to analysis (i.e., a “test liquid”).

Accordingly, the development of measurement devices that are reliable enough to operate for significant periods of time without maintenance (e.g., resistant to clogging, freezing, and fouling) and which are capable of supporting suitably accurate assessments of gases in various liquids, including the waters of oceans, lakes, rivers, and streams, is useful for environmental, industrial, and residential purposes.

### SUMMARY

Systems and methods for determining the concentration of gases in liquids are provided. The systems include an apparatus (equilibrator) having a high surface area that permits gases present (e.g., dissolved) within the liquid to diffuse into an exchange gas, permitting measurement of the gases. The systems find use in the measurement of a variety of gases including carbon dioxide (CO₂), methane, radon, hydrogen sulfide, total trihalomethanes, sulfur hexafluoride, nitrous oxide, sulfur dioxide, hydrogen, chlorine and/or bromine and the like. The systems may also be used in the measurement of volatile organic and inorganic components such as solvent (e.g., acetone, methyl acetate, ethyl acetate, methyl ethyl ketone, methyl isobutyl ketone and the like). The systems are designed to resist clogging or fouling by suspended material in the liquids. The present disclosure also provides methods for rapidly determining the partial pressure of various gases and volatile components in liquids including CO₂ (pCO₂) in water. The systems and methods are particularly useful for measuring/monitoring pCO₂ in coastal waters and other bodies of water where pCO₂ can change rapidly and vary widely at sites that are in close proximity to each other. The equilibrators can be connected to a single or to multiple instruments or the sensor partitions of instruments to measure one or more (multiple) gas species simultaneously, including but not limited to instruments such as gas chromatographs, mass spectrometers, and instruments that perform absorption spectroscopy such as non-dispersive infrared gas analyzers, fixed wavelength infrared detectors, laser absorption spectroscopes, cavity ring-down spectroscopes, and the like.

In addition to their use in coastal/environmental monitoring, the gas-liquid falling film equilibration system described herein can be used in industrial and laboratory settings where liquid-gas equilibration is needed. The system may be used in a continuous sampling format where liquid to be analyzed is continuously drawn from a source (e.g., a lake, river, retention pond, and the like) and introduced into the apparatus for gas and/or volatile component analysis. Alternatively, apparatus incorporating the equilibrators described herein may be adopted for laboratory or field measurements of discrete samples. Because the solubility of various gases and volatile organic and inorganic substances varies with temperature, the systems described herein may be equipped with one or more temperature control mechanisms to regulate the temperature at which the equilibration reaction takes place.

The present disclosure describes gas-liquid equilibration apparatus comprising:
a chamber c comprising an outer wall w that is disposed substantially symmetrically about a central axis, the outer wall defining an interior surface of the chamber, an exterior surface of the chamber, and space within the chamber;
an equilibration member em within the chamber having an equilibration member outer surface, an axis of rotation, and a bisecting plane perpendicular to the axis of rotation positioned at the midpoint of the equilibration member's axis of rotation;
the equilibration member being positioned within the chamber such that its axis of rotation and the central axis of the chamber coincide or substantially coincide;
the chamber, the exterior surface of the chamber, the interior surface of the chamber, the equilibration member and its outer surface, and the space within the chamber being divided into an upper portion above the bisecting plane and a lower portion below the bisecting plane;
the space within the upper portion of the chamber being in liquid (fluid) and gas communication with the space within the lower portion of the chamber via one or more gaps between the equilibration member and the interior surface of the chamber;
at least one (i) temperature control mechanism and/or (ii) sample injector 50;
a liquid inlet 1 located in the upper portion of the chamber positioned such that a liquid introduced into the chamber through the liquid inlet contacts the upper portion of the equilibration member outer surface;
a liquid outlet 2 located in the lower portion of the chamber through which some or all of liquid introduced into the chamber that collects in the lower portion of the chamber (e.g., by gravity) may exit the chamber as outflow;
a gas inlet located in the wall of the lower portion of the chamber; and
a gas outlet located in the wall (e.g., in the removably-resealable portion of the chamber) of the upper portion of the chamber;
wherein at least a section of the upper portion of the chamber wall is removably-resealable to the remainder of the upper portion of the chamber wall (e.g., the upper surface) and/or the outer wall.

This disclosure also provides for methods of determining the amount of one or more gases and/or volatile substances of interest present in a liquid (e.g., a liquid subject to analysis or “test liquid” or a liquid with a known level of gas or volatile substance or “calibration liquid”) using an apparatus as described hereinabove or an apparatus of any one of aspects 1-59 set forth in the disclosure that follows) comprising the following steps:
i) providing an apparatus of any one of aspects 1-59;
ii) introducing the liquid at an initial temperature (T1) into the chamber of the apparatus by way of the liquid inlet beginning at an initial time (t1) of a first time period such that it passes over the equilibration member thereby forming a falling film over all or part of the equilibration member's surface, and exits the apparatus by way of the liquid outlet;
iii) continuing the introduction of the liquid during the first period of time while optionally operating the temperature control mechanism to stabilize (maintain) the temperature of the liquid in a range that (a) encompasses the initial temperature (T1), (b) is in a range above the initial temperature, or (c) is in a range that is below the initial temperature;
iv) during the first period of time, directing a carrier gas into the chamber of the apparatus by way of the gas inlet such that it flows over the equilibration member in a direction that is counter current or substantially counter current to the flow of the liquid and exits the chamber of the apparatus by way of the gas outlet (“the gas that exits the chamber”);
v) during all or part of the first period of time, directing all or part of the gas that exits the chamber to a sensor (e.g., the sensor of an analytical instrument capable of detecting and/or measuring the amount of the one or more gases and/or one or more volatile substances); and
vi) determining the amount of the one or more gases and/or one or more volatile substances present in the liquid during the first period of time based on the output of the sensor.1

One or more measurements (e.g., a series of measurements) may be made over extended periods (e.g., more than a day, more than a week, or even more than a month) with samples drawn from bodies of liquids (e.g., lakes, oceans, rivers, retention ponds, etc.) particularly where the equilibrator is employed for remote operation given the equilibrator's resistance to fouling and blockage (substantially unattended/automated) during operation for such periods. The concentration of the gas (or gases) and/or volatile substance(s) of interest in the gas exiting the chamber can be used to determine the amount of the gas (or gases) and/or volatile substance(s) of interest present in the liquid according to Henry's law, based on the output of a detection system (i.e., sensor and any associated analytical instrumentation).

When employed for the measurement of gases and/or volatiles in discrete samples apparatus comprising the equilibrators described herein may be modified to recirculate a carrier liquid (e.g., water) in which the sample may be dispersed or dissolved (e.g., the sample is miscible or partly miscible) in a closed or substantially closed loop (a carrier liquid loop) that includes the equilibration chamber c, a sample injector 50, and a liquid reservoir 60 (for the carrier liquid) and any tubing and fittings connecting them. For example, the loop may comprise the liquid inlet 1, chamber c, the liquid outlet 2, sample injector 50, and liquid reservoir 60. The carrier gas is similarly recirculated through a closed or substantially closed loop (a carrier gas loop) comprising the gas inlet 3, chamber c, gas outlet 4, and headspace 63 (the gas filled space above liquid in the liquid reservoir), and any tubing connecting them. The loop recirculating the carrier gas may also include the path of carrier gas flowing through any equipment for conditioning the recirculating carrier gas (e.g., removing excess water vapor) and any sensors used to detect the gas(es) or volatile(s) of interest. Carrier gas and carrier liquid loops are considered closed or substantially closed when they do not permit either a loss or gain (e.g., external contamination) of the carrier gas or carrier liquid to a degree that will adversely affect assessment of an analyte of interest.

A method of determining the amount of one or more gases and/or one or more volatile substances present in a sample of test liquid may comprising the steps:
i) providing an apparatus comprising an equilibrator as described hereinabove (or an apparatus of any one of aspects 34-59 set forth in the disclosure that follows) wherein (a) a volume of carrier liquid is recirculated through a closed or substantially closed carrier liquid loop comprising the liquid inlet 1, chamber c, the liquid outlet 2, sample injector 50, and liquid reservoir 60, while optionally operating a temperature control mechanism to stabilize (maintain) the temperature of the liquid in a temperature range, and (b) a carrier gas is recirculated through a closed or substantially closed carrier gas loop comprising the gas inlet 3, chamber c, gas outlet 4, and headspace 63 of the liquid reservoir in a direction that is counter current or substantially counter current to the flow of the carrier liquid, and exits the chamber of the apparatus by way of the gas outlet 4;
ii) introducing a sample of the test liquid (a sample of discrete known volume) into the carrier liquid by way of a sample injector 50, the sample and carrier liquid entering the chamber c of the apparatus by way of the liquid inlet 1 such that they pass over the equilibration member thereby forming a falling film over all or part of the equilibration member's surface, and exit the apparatus by way of the liquid outlet 2;
iii) directing all or part of the gas that exits the chamber through gas outlet 4 to one or more sensors that produces an output corresponding to the amount of at least one of the one or more gases and/or one or more volatile substances present in the liquid (e.g., the sensor(s) of an analytical instrument capable of detecting and/or measuring the amount of the one or more gases and/or one or more volatile substances); and
iv) based on the output of the one or more sensors, determining the amount of the one or more gases and/or one or more volatile substances present in the sample of the test liquid.

Unlike the analysis of samples continuously drawn from a large natural, engineered, or artificial water body or source, the analysis of discrete samples (samples of a limited size) using the equilibrators described herein requires adaptations including a recirculating loop of carrier liquid. Instances when a discrete water sample may be used to measure dissolved gases or volatile compounds include those where: (i) there is not adequate liquid sample (e.g., water) volume to continuously sample (as described above) the liquid, (ii) the act of equilibration will change the bulk concentration of the analyte of interest (“the Observer Effect), or (iii) the concentrations of analyte are so high that they exceed the analyte sensor's specified working range if sampled continuously. In such circumstances, a discrete sample can be measured, but the volumes of carrier liquid and carrier gas, and thus the carrier liquid:carrier gas ratio and/or amounts, should remain substantially constant to avoid altering the system's internal pressure, which can adversely affect sensor measurement. To prevent carrier liquid:carrier gas ratio alteration and adverse pressure changes, the closed or substantially closed loop for recirculating of carrier liquid may be outfitted with a sample injector that compensates for volumes of carrier liquid (e.g., water) added or subtracted from the equilibrator/sensor system when a liquid sample is introduced. The injector may comprise a section of self-healing tubing, a self-healing septum, or a sample loop (also referred to as an injector loop or an injector sample loop) where an equal volume of carrier liquid in the carrier liquid loop is displaced by a sample to be injected (see, e.g., 53 in FIG. 8E).

An equilibrator apparatus of the present disclosure may also be connected to a closed or substantially closed carrier gas loop that is equipped with one or more ports (see, e.g., ports 19, 20, 21, and 22 in FIGS. 8A-8D). Those ports permit, among other things, the introduction of gas samples for analysis or gas standards for calibration while withdrawing an equal amount (e.g., volume) of gas to maintain the carrier gas loop's pressure and/or the carrier liquid:carrier gas ratio. Gas samples may also be withdrawn from those ports for analysis on instruments independent of (unconnected to) the equilibrator apparatus. A sample injector in the carrier gas loop using a sample loop like that shown for liquid samples in FIG. 8E may also be used for introducing gas samples into or extracting gas samples from the carrier gas stream.

Apparatus comprising an equilibrator of the present disclosure may comprise both a closed carrier gas loop and closed carrier liquid loop outfitted with sample injectors and/or ports (see, e.g., FIGS. 8B-8D). Those injectors and/or ports permit introduction of gas and/or liquid samples while compensating for volumes of carrier liquid (e.g., water) or gas added or subtracted from the apparatus (e.g., equilibrator/sensor system).

An apparatus comprising a gas-liquid falling film equilibration system configured for discrete sample analysis with closed or substantially closed carrier gas and carrier liquid loops allows the introduction of a known volume of liquid sample to the carrier liquid for analysis of a gas or volatile component. In the analysis, the instrument's carrier liquid reservoir is filled with an appropriate carrier liquid (e.g., tap water, distilled water) and the space occupied by gas is filled with a suitable carrier gas (e.g., air). The instrument is operated with carrier gas and carrier liquid circulating (recirculating) until the carrier liquid has reached a stable equilibrium with the carrier gas for the analyte of interest; this is the starting condition. As the liquid sample of known volume is injected, an equal volume of carrier liquid may be removed without contamination from surrounding atmosphere (e.g., using an injector sample loop as in FIG. 8E). The system is allowed to run until a new stable equilibrium condition is reached. This measurement represents the diluted concentration value. Because the starting volume and concentration of carrier liquid is known, and the diluted volume and concentration are known, and because mass is conserved, the undiluted sample concentration can be determined by back calculation. All partial pressures are converted to molarity values via Henry's Law Solubility constant for the solute and solvent (e.g., water and CO₂ or CH₄) at measured temperature and 1 atm. Once in molar form, the dilution calculation may be carried out: (m1×v1)+(m2×v2)=(m3×v3), where m1 and v1 are mass and volume of starting conditions, m3 and v3 are diluted mass and volume and m2 and v2 equal the sample mass and volume.

An apparatus comprising a gas-liquid falling film equilibration system described herein configured for discrete sample analysis with closed or substantially closed carrier gas and carrier liquid loops may be employed without using injectors/ports that permit maintenance of the carrier liquid:carrier gas ratio and/or carrier gas pressure. Used in that fashion, the amount of analyte may be determined using empirically derived standard curves or by back calculations that correct for changes in volume and/or pressure while assuming the values of various parameters and the behavior of the system complies with the applicable gas laws (e.g., Henry's law). However, use without compensation for changes in volume and pressure introduces potential variation in the precision and/or accuracy of the measurements.

Henry's law describes the equilibrium ratios of substances distributed across their aqueous/liquid and gas phases in dilute solutions. Partitioning of any gas phase analyte into a liquid is uniquely affected by temperature and the liquid. Because the gases and volatile substances frequently display non-linear solubility behavior in liquids with regard to temperature, the effect of heating on Henry's law constants (KH, also called the air-water partition coefficient) is complex and difficult to predict. This uncertainty is exacerbated when solutes/analytes are constituents of heterogeneous mixtures in non-pure solvents (e.g., seawater, groundwater, waste streams). Although it might be argued that merely increasing the temperature at which an equilibrator operates will increase the rate the system reaches equilibrium, elevating the temperature may result in inaccurate and/or imprecise measurements of dissolved gases and/or volatile substances due to shifts in the position of the equilibrium reflected in temperature-related changes to the Henry's law constant. For example, increasing the temperature of a gas-liquid system may increase the rate at which gases and other volatile substances dissolve in a liquid and equilibrate with a carrier gas phase (effectively decreasing the rate constant of the instrument's response). However, because gases and/or volatile analytes may display complex solubility behavior in the liquid phase as temperature changes, the precision and/or accuracy of their measurement using equilibrated gas and liquid phases may be impacted when the analysis requires compensation for changing temperatures. In addition, the potential for inducing chemical reactions that consume or produce an analyte gas or volatile substance when shifting the temperature away from the ambient temperature also cannot be ignored.

Measurements of gases and volatile substances in test liquids are further complicated where two or more of the gases or volatile substances being measured behave differently (e.g., display significantly different shifts in solubility or equilibrium ratios with the test liquid) when the measurement temperature is shifted. Put another way, because gases and other volatile substances (analytes) in test liquids do not react uniformly to changes in the temperature, the effect of changing the temperature at which measurements are conducted away from the liquid's ambient temperature, including heating to increase the response rate of the equilibrator, are inherently unpredictable. Moreover, raising the temperature of equilibration reaction will generally increase the equilibrium of the vapor pressure of the test liquid. The vaporized liquid may interfere with the detection of the gases of interest including by condensing in the detection system (e.g., in the sensor) or the lines (tubing) connecting the equilibrator to the sensor (where detection is optical, the vapor may also alter sample absorbance).

Accordingly, altering the temperature of equilibrator operation away from the ambient temperature, including raising the temperature to increase the response rate of the equilibrator, can negatively impact the accuracy of measurements. That unpredictability is exacerbated when one or more gases and/or volatile solutes are constituents of heterogeneous mixtures in non-pure solvents (e.g., seawater, groundwater, sewage, industrial waste streams). The temperature control mechanism(s) of the equilibrator described herein permits temperature regulation (temperature control) by providing heating or cooling of the test liquid or carrier liquid into which samples of a test liquids may be introduced. The apparatus may also include mechanisms to compensate for the effects of temperature regulation (e.g., to limit condensate reaching the detector system(s), and particularly their sensor(s), that are used to measure the analyte gases or volatile substances present in the test liquid).

The apparatus may be used to determine the concentration of a variety of gases and/or volatile substances in a diverse number of liquids, including the concentration of carbon dioxide and/or volatile hydrocarbons (e.g., methane) in aqueous systems such as bodies of fresh or salt water. The apparatus can also be used to measure other volatile substances (e.g., H₂S and volatile solvents such as methyl acetate, ethyl acetate, acetone, dichloromethane, and the like) in aqueous systems (e.g., fresh or salt water). Tests of the falling film liquid-gas equilibrators described herein across broad ranges of gas (e.g., CO₂) concentrations and liquid (e.g., water) and carrier gas (e.g., air) flow rates indicate that falling film equilibrators as described herein have the ability to produce consistent, precise, and accurate dissolved gas measurements (e.g., dissolved pCO₂ measurements) even across significantly different equilibrator dimensions.

The equilibrator may be used to assess gases and other volatile substances in liquids at ambient temperatures or, where the precision and/or accuracy of measurement require more precise control, the temperature of the equilibration reaction may be controlled (i.e., applying heating or cooling) as needed for the measurements.

Temperature control of the equilibrator and temperature monitoring can be accomplished in various ways including, but not limited to, heating or cooling the liquid coming into the equilibrator (e.g., test liquid or carrier liquid circulating in carrier liquid loop) upon which a measurement is made, heating or cooling the equilibration member, heating or cooling the incoming carrier gas, and/or heating or cooling the chamber itself. Accordingly, the equilibrator may be outfitted with one or more temperature control mechanisms to effect changes to the temperature of the test liquid introduced into the equilibrator's chamber or carrier liquid recirculating in the carrier liquid loop, the temperature of the equilibration member, and or the temperature of the incoming carrier gas. Generally, temperature can be regulated using one or more temperature sensors to effect thermostatic control. For example, one or more temperature sensors may be placed to measure the temperature of the equilibration member or its surface. Insulation or heat exchangers can be applied to or incorporated into, for example, the chamber or liquid inlet control element 27 as necessary.

When making measurements in systems where the equilibration temperature is shifted from the ambient temperature of the test liquid, and particularly where the liquid is heated to increase the volatilization of the analyte and/or the rate of equilibration, the measurements may require compensation for accuracy and/or precision of the measurement. Accordingly, when using the equilibrator with the application of heat to enhance equilibration, the temperatures of the ambient and heated or cooled liquid (e.g., test liquid) should be measured, recorded (e.g., at least the differential temperature recorded), and used to correct partial pressure and dissolved concentration determinations.

### BRIEF DESCRIPTION OF THE DRAWINGS

FIG. 1 shows an illustration introducing the general terminology for the falling film gas liquid equilibrators described herein using an equilibrator with a substantially cylindrical chamber having a height h at the central axis cax ( [symbol] ), an inner radius r, an outer radius R, and a wall w of thickness t. The equilibrator is shown as having a substantially spherical equilibration member em with an axis of rotation axr( [symbol] ) having a length substantially equal to the height of the em, and a maximum radius emr, appearing, in this instance, at the equator e of the sphere. As the emr is less than the inner radius r, a gap g is shown between the inner surface of the wall w and the surface of the em. In the illustration the cax and the axr are substantially aligned. The drawing is not to scale.

FIG. 2A shows a schematic cutaway of an equilibrator with a substantially cylindrical chamber with a wall w showing its spherical equilibration member em and a gap g between w and em. The schematic shows the liquid inlet 1, liquid outlet 2, gas inlet 3, and gas outlet 4. In the figures, liquid is denoted by the wavy lines [symbol] and gas by the dashes [symbol] Arrows indicate the direction of flow.

FIG. 2B shows a schematic cutaway of an equilibrator with a substantially cylindrical chamber with a wall w showing its spherical equilibration member em and a gap g between w and em. The schematic shows the liquid inlet 1, liquid outlet 2, an optional heat exchanger for the carrier gas illustrated by a Peltier thermoelectric panel 29 (cooled side facing the front, heated side facing the back), gas inlet 3 (dashed tube) on the back (heated side) of the thermoelectric sheet, gas outlet line 4 on the front (cooled) side of the thermoelectric sheet, and an optional condensate return line 4a. In the figures, liquid is denoted by the wavy lines [symbol] and gas by the dashes

[symbol]

FIG. 2C shows a schematic cutaway of an equilibrator as in FIG. 2B with inlet 1 connected to a liquid supply 24 having a counter current heat exchange segment 26 that recovers heat from liquid exiting the chamber through outlet 25. FIG. 2C also provides for an optional liquid inlet temperature control element (e.g., a heater) 27 to regulate the temperature (e.g., heat) the liquid entering the equilibrator at inlet 1 and a gas inlet heating and/or cooling element 28 for heating or cooling gas entering the equilibrator via inlet 3.

FIG. 2D shows a schematic cutaway of an equilibrator as in FIG. 2B but sectioned through the equilibration member to show its heat-exchange fluid filled interior 30. Heat-exchange fluid input line 31 and return line 32 permit circulation of the heat-exchange fluid with the heating and/or cooling unit 33. The heat-exchange unit is marked with the symbol ±A to indicate that it may provide heated or cooled heat-exchange fluid. One or more independently placed temperature sensors 34 (e.g., thermometers and/or thermostats) are located in contact with the heat-exchange fluid and or the equilibration member. The apparatus may contain a pump (not shown) to cause circulation of the heat-exchange fluid.

FIG. 2E shows a schematic cutaway of an equilibrator as in FIG. 2B but sectioned through the equilibration member to show its heat-exchange fluid filled interior 30. Coolant (e.g., refrigerant or cooled aqueous liquids) input line 41 and coolant return line 42 permit cooling of the heat-exchange fluid by the cooling unit 40 through heat exchange occurring at cooling element 44. The cooling unit is marked with the symbol −Δ to indicate that it provides cooling. Heat may be provided by one or more heating elements 43, located in the heat exchange fluid and/or attached to the inner surface of the equilibration member. The heating element may be an electrical (e.g., providing electrical resistance heating) or attached source of heated fluid (connecting wires or tubing to external sources of electricity or heated fluid are not shown). One or more independently placed temperature sensors 34 (e.g., thermometer) may be located in contact with the heat-exchange fluid and/or the equilibration member.

FIG. 3 shows a schematic cutaway of an equilibrator with a substantially cylindrical chamber and a spherical equilibration member as in FIG. 2. As diagramed, the planar upper surface of the cylinder 5 has a lip 6 and is removably-resealable to (against) the cylindrical wall, at location 7 (via e.g., an O-ring). In such an embodiment, the planar upper surface acts like a substantially air-tight/water-tight “lid” on the cylindrical chamber. The annular support (as) supporting the equilibration member is shown below the level of liquid (e.g., water) in the chamber.

FIG. 4 shows a schematic cutaway of an equilibrator with a substantially ellipsoidal chamber and an ellipsoidal equilibration member. Holes, gaps, or channels in the annular support as permit liquid introduced through inlet 1 to reach the liquid outlet 2.

FIG. 5 at a-n shows generalized cross sections of equilibration members including: a spherical, b ellipsoidal, c ovoidal, d fusiform shape, e hemispherical, f hemiellipsoidal, g hemiovoidal, h domed frustum (domed frustoconical section), i domed vertical right cylinder, j column having oscillating sides (e.g., sinusoidal changes in the column radius), k conical having sinusoidal oscillating sides (column with sinusoidal changes in the column radius), l a series of spheroids or discs, m four spheroids, and n a series of spheroids or discs of increasing size.

FIG. 6 at a-o shows generalized cross sections of equilibrators incorporating the equilibration members shown in FIG. 5. Each equilibration member is shown in an equilibrium chamber having a liquid inlet 1, a liquid outlet 2, a gas inlet 3, and a gas outlet 4 indicated by arrows.

FIGS. 7A and 7B show in 7A a gas inlet nozzle 100, and in 7B a gas outlet with a shield 101. The inlet and outlet are shown in a portion of chamber wall 102 and are held in place by retaining nuts 103 that engage threaded sections 104. When fully tightened, nuts 103 cause compression of seals 105 providing substantially gas and liquid tight seals. The nozzle shown in FIG. 7A has an internal pathway 110 through which gas may enter the chamber and be dispersed through nozzle end 120. The gas outlet shown in FIG. 7B has a shield section 101 that prevents droplets of liquid (e.g., water) from entering the entrance of the gas outlet 130, which is in gas communication with the internal passage 140 that forms part of the gas outlet.

FIG. 8A shows a schematic of one configuration of a system that incorporates equilibrator E. Liquid supplied to the liquid inlet 1 may be from an open source, such as a lake, stream or river, and the liquid exiting the equilibrator chamber via outlet 2 may be returned to the open source. Alternatively, where discrete samples are to be assayed for gas or volatile content, the liquid entering the equilibrator chamber via inlet 1 and exiting via outlet 2 may be recirculated to a reservoir in a closed or substantially closed carrier liquid loop as illustrated in FIGS. 8B, 8C, and 8D. Various components of the system may be connected by wires or wireless communication components that are not shown. In addition, sensor(s) 16 need not be encased in the analytical instrument.

FIGS. 8B, 8C, and 8D show a schematic of systems incorporating equilibrator E that may be utilized for discrete sample analysis where samples are introduced via injector 50. In each of FIGS. 8B-8D, the equilibrator E is in fluid communication with a reservoir 60 that receives liquid exiting chamber c via outlet 2, and returns liquid to the chamber via liquid inlet 1. In FIGS. 8A-8C the temperature of liquid in the reservoir 62, and accordingly the chamber, may be regulated by a temperature control element 61 that may be connected to a temperature sensor 34 for temperature regulation. In FIGS. 8C and 8D reservoir chamber c is located vertically above reservoir 60 and receives liquid via a liquid outlet 2 located in the wall w of the chamber (FIG. 8C) or in the lower surface of the chamber 45 (FIG. 8D).

FIG. 8E shows an injector 50 of six port design employing a fixed volume sample loop 53. The injector is shown with hub 56 rotated to the sample loading position at A and to the sample injection position at B.

FIG. 9 shows a performance comparison of an equilibrator having an 8-inch diameter spherical equilibration member with an equilibrator having a 10-inch diameter equilibration member over a 6-day period in a dynamic pCO₂ test as described in Example 1. Measurement values taken from the equilibrator having an 8-inch diameter spherical equilibration member are shown as a filled dot “●”, and those taken with the equilibrator having a 10-inch diameter equilibration member are shown as an open circle “◯”. Similar results are obtained down to about 3.7-inch diameter spherical equilibration members.

FIGS. 10A-10F show six drawings of a spherical falling film equilibrator apparatus of the type shown schematically in FIG. 3 and used in Example 1 (10-inch diameter equilibration member) having a 13.25 liter chamber that is substantially a virtual right cylinder (VRC): FIG. 10A, fully assembled for operation; FIG. 10B, opened to show the equilibration member with the view from above; FIG. 10C, showing a rubber or plastic seal along the upper edge and an annular support within the chamber, with the gas inlet visible in the photo about 4 o'clock on the chamber wall; FIG. 10D, the equilibrator with a 3.7-inch diameter equilibration member and a 0.565 liter chamber; FIG. 10E, side by side comparison of a 6-inch diameter equilibration member and a clear 4 liter chamber (left) and an 8-inch diameter equilibration member and a 7.57 liter chamber (right); FIG. 10F, side by side comparison of a 3.7-in diameter equilibration member in a clear 1 liter chamber (left) and an 8-inch diameter equilibration member in a 7.57 liter chamber (right).

FIG. 11 shows the measurement of CO₂ and methane in the water of a tidal salt marsh creek connected to the Chesapeake Bay in Maryland, U.S.A., along with the water height for the period of December 24th-30th of 2021.

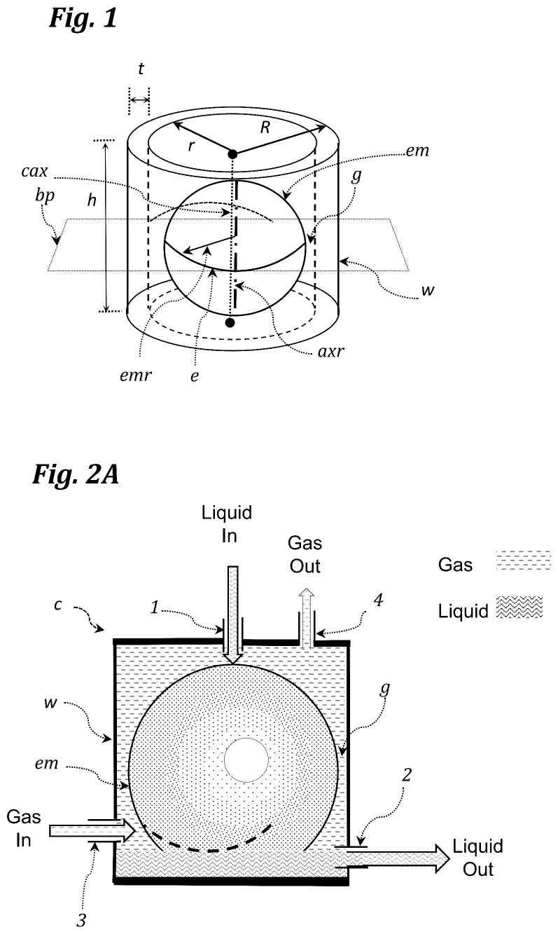

### DETAILED DESCRIPTION

### Definitions

An equilibrator is an apparatus for contacting a gas and a liquid so as to exchange one or more gases between the phases. The term equilibrator does not mean the apparatus brings the two phases (gas and liquid) necessarily into perfect equilibrium, but rather brings the phases to a state approaching equilibrium or a dynamic equilibrium so that the amount and/or relative changes in the amount of gases/volatile materials in the liquid can be determined.

A liquid inlet is a point in the surface of the chamber wall where liquid enters the chamber. The liquid inlet may terminate at or be in the form of a nozzle.

A nozzle is an extension or projection at the gas inlet or liquid inlet that directs the flow of gas or liquid within the chamber. Liquid inlets have an opening with a minimum inner diameter to avoid plugging and promote complete wetting of the equilibration member, thereby optimizing the generation of a falling film gas exchange surface.

“Amount” of a gas or gases as used herein may be expressed by any suitable measure including concentration in the form of molarity, weight per volume (e.g., volume of carrier gas), volume/volume (e.g., per volume of carrier gas, or percent volume of carrier gas), partial weight or mass (grams gas of interest/gram of gas or liquid, such as ppm by weight), part per million by volume (ppmv), or partial pressure.

Calibration gas or calibrator gas is a gas having a known amount of the gas of interest.

Carrier gas as used herein is a gas, other than the gas of interest, which is passed through the equilibrator and into which the gas of interest diffuses, and which may be subject to analysis to determine the amount of the gas of interest present.

The term, “removably-resealable,” as used herein means capable of being removed from a location on an object (e.g., the equilibrator chamber wall) and replaced in that location to form a seal. More specifically, with regard to a section of the chamber wall, removably-resealable means that a section of the chamber wall can be removed to provide access to the interior of the chamber and then replaced and sealed sufficiently to the remainder of the chamber wall to permit operation of the apparatus (e.g., without loss of carrier gas or liquid from the chamber that would interfere with its operation).

Ellipsoidal as used herein means having the form of an ellipsoid.

Ovoidal as used herein means having the form of an ovoid (e.g., egg shaped).

Spheroidal as used herein means having the form of a sphere or spheroid.

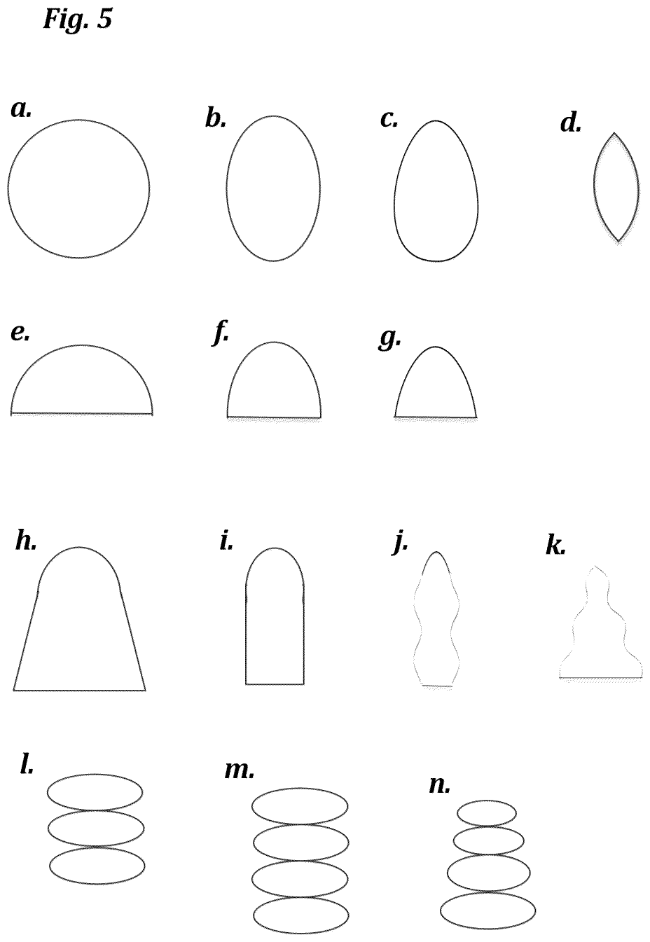

Vertically stacked equilibration member(s) means equilibration members formed from a series of elements having an axis of rotation that when aligned vertically in the equilibration chamber each have their axis of rotation substantially aligned with the central axis of the equilibration apparatus. See, e.g., FIG. 5 at l to n and 6 at l to n.

### DESCRIPTION

The measurement of CO₂ and other gases or volatile materials present in liquid (e.g., aqueous) samples may be conducted using a variety of techniques. In many techniques the gas(es) of interest are removed/forced out of the liquid for measurement in the gas phase. The gas phase may include a carrier gas or mixture of carrier gases into which the gas(es) of interest in the liquid moves (e.g., exchange or are added to the carrier gas(es)). The movement of gases out of the liquid may be accomplished by a number of processes including, but not limited to, alteration of the chemical composition of the liquid (e.g., acidification), reduction of the pressure, and passive diffusion. A variety of different equilibration apparatus or “equilibrators” has been developed with the goal of efficiently exchanging/equilibrating the gases in the liquid phase with a carrier gas that is in turn directed to the sensor of a detection/analysis instrument (gas analyzer) for measurement of the gas(es) of interest. Among the equilibrator designs are the “shower type”, “bubble Weiss type”, and “laminar flow type” described by Frankignoulle et al. (Water Res. Vol. 35, No. 5, pp. 1344-1347, (2001)). While each of such systems may be useful, they suffer from a variety of disadvantages including, but not limited to, the inability to handle materials with suspended particles, susceptibility to fouling (e.g., biofouling), difficulty in removal of deposits (cleaning) built up by suspended particles and/or fouling, and instability when subject to tipping or motion during measurement.

The present disclosure describes, and provides for the use of, a falling film type of equilibrator that provides a rapid response time that is governed by the dead time (i.e. time after a change to the input before its initial detection) and the lag time (i.e. how fast the equilibration/detection process proceeds), the specific values of which depend on the specifics of the equilibrator design and the detection instrument that is being used. The time constant tau (τ), also known as the e-folding time, is the time necessary for an instrument to respond to an induced step change. τ=the 1/e decay in concentration (n τ=time at which Cₜ/C₀=1/eⁿ⁻; e.g., 3τ=time when Cₜ/C₀=1/e³) when a step change is from high to low. Conversely, when the step change is from low to high concentrations, the response is given by n τ=time when Cₜ/C₀=1-1/eⁿ. For spherical falling film equilibrators described herein (e.g., with equilibration member diameters of around 3.5 to 10-inch diameters), τ˜3 minutes and 3τ (i.e., to reach 95% response) ˜8 minutes for small diameter equilibrators for carbon dioxide steps from about 100 ppmv to about 50,000 ppmv. For an equilibrator having a VRC chamber with a volume of about 7.57 liters and a spherical equilibration member about 8 inches (20 cm) in diameter operated at a water flow rate in the range of 225-380 liters per hour and a one (1) liter/minute carrier gas (air) flow rate, r can be as low as about 3 to 4 minutes, although it may be longer (e.g., about 4 to about 6 minutes, about 6 to about 8 minutes, or about 8 to about 9 minutes) depending on the particular operating conditions. The dead time (time from the initiation of the step change in dissolved gas until the sensors first respond) for such an equilibrator operated under the same conditions is generally less than about 1 minute. For equilibrators where the chamber headspace has been minimized, the response time for carbon dioxide measurements may be less than 3 minutes (e.g., less than 2.5, 2.0, 1.5 or 1.0 minutes, or in a range from 1.0-3.0 minutes, 1.0-2.0 minutes, or 2-3 minutes). Similarly, dead times can be less than one (1) minute (e.g., less than 50 seconds, 40 second, 30 seconds, or 20 seconds, or in a range from 20 seconds to 1 minute, 20-40 seconds, or 40 seconds to 1 minute).

In a first embodiment the equilibrator comprises a chamber c formed of a wall w having a liquid inlet 1 and liquid outlet 2 subject to measurement; an equilibration member em enclosed within the chamber; and a gas inlet 3 and gas outlet 4 for a carrier gas such that the carrier gas flows counter current or substantially counter current to the flow of liquid through the chamber. Gas flow substantially counter current to the flow of liquid through the chamber means that the flow of gas may not be counter current at all points (e.g., the gas may swirl or form eddies in the chamber) but there is a net flow of gas moving upward in the chamber as the liquid moves downward over the equilibration member due to the action of gravity. The equilibration member has a surface over which the liquid can form a film over all or part of the surface area (e.g., over greater than 50, 60, 70, 80, 90, or 95% of its surface area) where flow is not inhibited or impeded by the shape or the design or equilibrator orientation). In such an embodiment the equilibration member may be wettable by the liquid (e.g., the equilibration member is hydrophilic, and the liquid is aqueous). In embodiments, the equilibration member is hydrophilic and the contact angle of the equilibration member with water is less than about 90°, less than about 80°, less than about 70°, less than about 60°, less than about 50°, less than about 40°, less than about 30°, less than about 20°, or less than about 10°, as measured by a goniometer at 22° C.

In an aspect of the first embodiment (second embodiment) the equilibrator comprises:
a chamber comprising an outer wall that is disposed substantially symmetrically about a central axis, the outer wall defining an interior surface of the chamber, an exterior surface of the chamber, and space within the chamber;
an equilibration member within the chamber having an equilibration member surface, an axis of rotation, and a projected bisecting plane bp that is perpendicular to the axis of rotation, the bp positioned at the midpoint of the equilibration member's axis of rotation (e.g., where the em is a sphere the bp would pass through its equator);
the equilibration member being positioned within the chamber such that its axis of rotation and the central axis of the chamber coincide or substantially coincide (e.g., substantially align);
the chamber, the interior and exterior surface of the chamber, the chamber wall, the equilibration member within the chamber, and the space within the chamber being divided into an upper portion above the position of the bisecting plane and a lower portion below the bisecting plane;
the space within the upper portion of the chamber being in liquid and gas communication with the space within the lower portion of the chamber via one or more gaps between the equilibration member and the chamber wall;
a liquid inlet located in the upper portion of the chamber positioned such that a liquid introduced into the chamber from the liquid inlet contacts the portion of the equilibration member located in the upper portion of the chamber;
a liquid outlet located in the lower portion of the chamber positioned to permit outflow of some or all of the liquid and suspended solids (e.g., sediments, detritus, phytoplankton, etc.) introduced into the chamber that tend to collect in the lower portion of the chamber by gravity;
a gas inlet located in the wall of the lower portion of the chamber; and
a gas outlet located in the wall of the upper portion of the chamber.

During operation the liquid (e.g., water) draining out of the outlet forms a seal such that gas may not enter or exit the chamber. Importantly, this seal will self-correct internal pressure to match ambient atmospheric pressure (i.e., if either positive or negative pressures begin to develop inside the equilibrator chamber, the seal will be momentarily broken, allowing inside and outside pressure to equalize, with little or no effect on carrier gas-liquid (e.g., air-water) equilibration. In such an embodiment, at least a section of the upper portion of the chamber wall may be removably-resealable to the upper portion of the exterior surface of the chamber and/or the upper portion of the chamber wall.

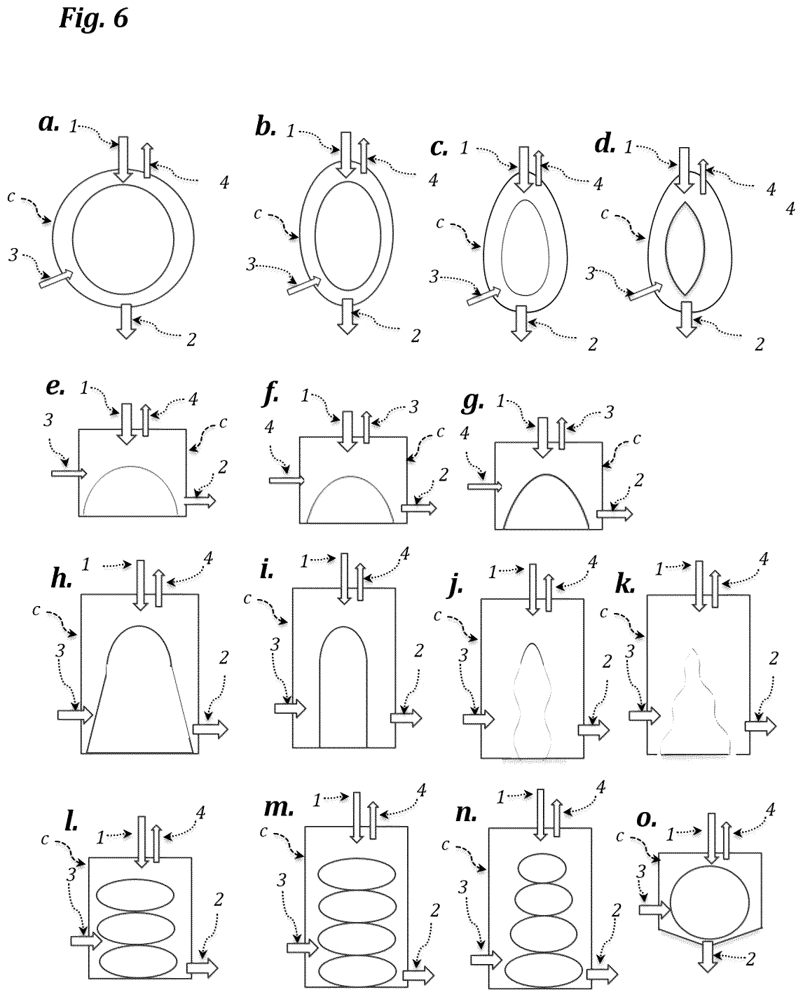

In an aspect of the first embodiment (a third embodiment) the equilibrator comprises:
a chamber comprising an outer wall that is disposed substantially symmetrically about a central axis, an upper surface (e.g., an upper exterior surface), and a lower surface (e.g., a lower exterior surface), that together define an interior surface of the chamber, an exterior surface of the chamber, and space within the chamber;
an equilibration member within the chamber having an equilibration member surface, an axis of rotation, and a bisecting plane perpendicular to the axis of rotation (e.g., an equilibration member substantially spheroidal, ellipsoidal, ovoidal, or other shape discussed below, see, also, FIGS. 5 and 6), with the bisecting plane positioned perpendicular to the axis of rotation at the midpoint of the equilibration member's axis of rotation;
the equilibration member being positioned within the chamber such that its axis of rotation and the central axis of the chamber coincide or substantially coincide such that the bisecting plane of the equilibration member is substantially perpendicular to the central axis of the chamber;
the chamber, the exterior surface of the chamber, the chamber wall, the equilibration member within the chamber, and the space within the chamber being divided into an upper portion above the position of the bisecting plane and a lower portion below the bisecting plane;
a liquid inlet located in the upper portion of the chamber positioned such that a liquid introduced into the chamber from the liquid inlet contacts the portion of the equilibration member located in the upper portion of the chamber;
a liquid outlet located in the lower portion of the chamber positioned to permit outflow of some or all of the liquid and suspended solids introduced into the chamber that collect in the lower portion of the chamber by gravity;
a gas inlet located in the lower portion of the chamber; and
a gas outlet located in the upper portion of the chamber;
   wherein at least a section of the upper portion of the chamber is removably-resealable to the remainder of the upper portion of the chamber wall (e.g., upper surface) and/or the outer wall.

In such an embodiment, at least a section of the upper portion of the chamber wall may be removably-resealable to the upper portion of the exterior surface of the chamber and/or the upper portion of the chamber wall.

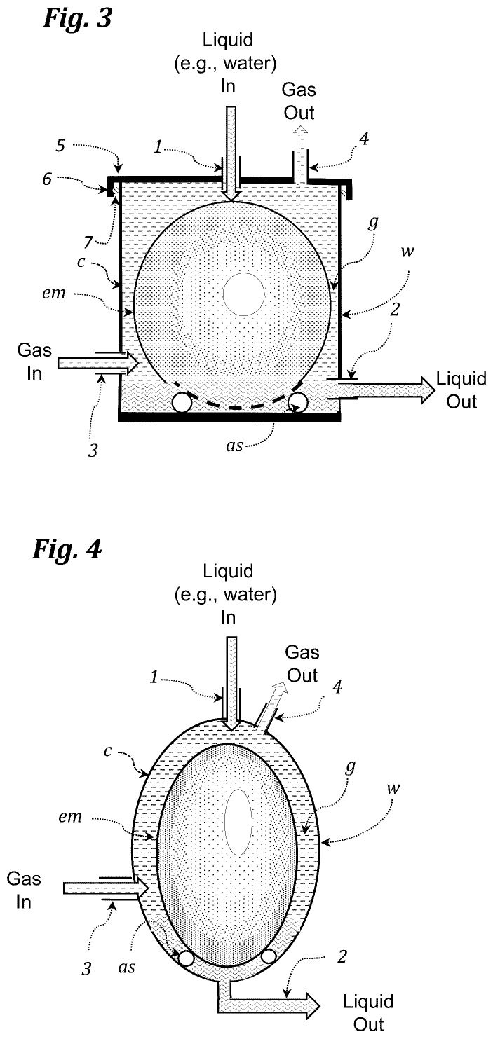

In any of the first, second or third embodiments recited above, the chamber may be a vertical right cylinder. In some embodiments, the section of the upper portion of the chamber wall that is removably-resealable to the upper portion of the exterior surface of the chamber, and/or the upper portion of the chamber wall, may be the planar upper surface of the cylinder 5 or a portion thereof. In such an embodiment, the planar upper surface may have a lip 6, which seals against the chamber wall at, for example, location 7, so that it acts like a “lid” on the cylinder of the chamber. The seal at location 7 in FIG. 3 is shown as an O-ring; however, other types of seals may be employed alone or in addition to O-rings, including compression seals and snap fit lids. See, e.g., FIG. 3. The liquid inlet 1 and/or gas outlet 4 may be positioned in the planar upper surface, and either or both may be positioned in a portion of the planar upper surface that is removably-resealable to the remainder of the chamber.

In other embodiments, including the first, second or third embodiments recited above, the chamber is not a vertical right cylinder. In such embodiments, the chamber may be a shape, such as an ovoid, ellipsoid, or spheroid, that more closely conforms to the shape of the equilibration member leaving a smaller chamber headspace volume that will shorten the overall response time of the equilibrator to changes in the gas content of the liquid introduced for sampling.

Various features and components of the equilibrators described herein are discussed in further detail below (e.g., the shape of the equilibration member and/or chamber, material for constructing the equilibrator, and placement of inlets and outlets).

The use of the falling film equilibrators having the above-mentioned designs, which are further described herein, and particularly those with spheroidal, ellipsoidal and ovoidal equilibration members offers a variety of advantages. Such equilibrators offer the ability to form and sustain a reliable and effective thin layer falling film. Because the design uses an equilibration member centrally located in the chamber, the orientation and/or disturbance of the equilibrator (e.g., placement on a non-level surface or movement on a boat or floating platform) is far less critical to its successful operation than falling films generated on other surface geometries such as vertical tubes or planar surfaces). This is especially true for embodiments where the equilibration member has a spheroidal, ovoidal, or ellipsoidal surface for falling film generation. This contrasts with vertical falling films used, for example, in industrial applications such as falling film evaporators that contain hundreds of individual vertical tubes that can be several stories tall. These systems are stationary as they need to remain plumb to the ground for effective use as they are prone to suboptimal flow or failure if disturbed or deviated from a vertical position. In addition, vertical tube equilibrators must be engineered and built to higher tolerances than the equilibrators described herein. Furthermore, the mechanism for introducing liquid at the tops of vertical tube-type equilibrators must be properly designed, and the flow carefully controlled, if sustained falling films are to be maintained.

In contrast, given adequate water flow rate, the falling film design described herein has the advantage of maintaining a sustainably wetted surface for gas exchange (e.g., fully wetted or greater than 50%, 60%, 70%, 80%, 90%, or 95% wetted), even when the water intake is tilted up to nearly 45° from vertical. Thus, physical disturbances and non-plumb placements do not affect the generation and maintenance of falling films and, by extension, do not disturb the proper function of the air-water equilibrator.

The equilibrators described and provided for herein (e.g., those with spherical, ellipsoidal, or ovoidal equilibration members) also offer some distinct advantages over shower, so-called marble laminary flow (laminar flow), bubbling, and membrane equilibrator designs. Each of those designs has narrow passages that are prone to clogging (blockage) and/or fouling (buildup of deposits). Clogging and fouling may have a variety of sources including sediments, suspended particles, deposition of minerals from the liquid, phytoplankton, detritus, biofouling by marine and/or aquatic organisms (e.g., barnacles, bryozoans, hydroids, etc.), and combinations of any two, three or more thereof.

Clogging and/or fouling can easily compromise water flow and operation of air-water equilibrators. For example, equilibrator designs that employ a showerhead to create water droplets/mists will cease to function with even minor clogging/fouling, as will equilibrators that employ air stones or frits (which foul from materials in the liquid) that are used to introduce carrier gases into bubbling equilibrators. Likewise, sediments and phytoplankton can clog the interstices among marbles in vertical laminary flow equilibrators, thereby compromising gas exchange. Trapped organic material and organisms can also promote biogenic processes that affect gas concentrations inside the equilibrator (e.g., respiration and photosynthesis). Clogging and biofouling greatly reduce the utility of these equilibrator designs, particularly where eutrophic and/or turbid aqueous samples are being analyzed. This includes eutrophic and/or turbid samples of water from coastal oceans, estuaries, lagoons, rivers, streams, lakes, reservoirs, and the like.

The falling film equilibrators described and provided for herein (e.g., those with spheroidal, ellipsoidal, and ovoidal equilibration members) use relatively large and difficult to clog water ports that provide unimpeded free flowing liquid (e.g., water) to form the falling film. As such, they avoid narrow channels or paths for liquid flow that are prone to blockage by clogging and fouling. In some embodiments, anti-fouling coatings (e.g., marine anti-fouling paint with, for example, copper incorporated) can be used on the surface of the equilibrator. In addition, the internal walls of the equilibrator chamber and fittings can be coated with anti-fouling treatments, coatings or paints to further prevent biofouling. In an embodiment at least the interior surface of the chamber is coated with a hydrophobic coating, or hydrophobic and oleophobic coating, that resists fouling. In addition, the nature of the liquid flow through the chamber tends to sweep/carry particulate matter off the equilibration surface and out of the chamber, preventing buildup.

In addition to being resistant to clogging and fouling, the spherical falling film equilibrators described and provided for herein (e.g., those with spheroidal, ellipsoidal, and ovoidal equilibration members) are comprised of a very few parts that may be made of durable materials that can withstand impact and/or exposure to the environmental conditions under which testing is conducted (e.g., durable plastics, or stainless steel). In embodiments described herein, the equilibrator comprises a section of the chamber that is easily removed, thereby opening the chamber and permitting the apparatus to be quickly cleaned by hand in the field. In an embodiment the section of the chamber wall that is removable is of sufficient size to permit the equilibration member to be removed. The removable section of the chamber wall is designed to be placed back in position and sealed to the remainder of the chamber (a removably-resealable section).

As the equilibrators described and provided for herein do not rely on small orifices, channels, or interstices for gas exchange and proper function, the cleaning and maintenance of the equilibrators are minimized and can be performed far less frequently than for the traditional air-water equilibrators described above. As such, the equilibrator design can be deployed in the field for much longer periods of time between maintenance checks.

### 1. Equilibration Members

The equilibration member, which is disposed inside of the chamber of the equilibration apparatus, provides a surface upon which the liquid subject to measurement (e.g., water or salt water) forms a film as it passes over the surface and is drawn downward by gravity. The equilibration apparatus described herein may employ equilibration members in a variety of shapes and sizes. The equilibration members are generally symmetrical about a central axis, which extends from the top to the bottom of the equilibration member, and as indicated below is used to center the member within the chamber. As a matter of locating, among other things, liquid inlet(s), liquid outlet(s), gas inlet(s) and gas outlet(s), the equilibration member may be understood to be divided into an upper portion and a lower portion by a bisecting plane that is projected substantially perpendicular to the central axis at the midpoint between the top and bottom of the equilibration member.

In various embodiments the shape of the equilibration member is substantially a spheroid, an ellipsoid, an ovoid, a fusiform shape, a hemisphere, a hemiellipsoid, a hemiovoid, a domed frustum, a domed column, a column having oscillating sides (e.g., sinusoidal changes in the column radius), a cone having sinusoidal oscillating sides (sinusoidal changes in the column radius), or a series of spheres or discs (two, three, four or more) aligned along a central axis. See FIGS. 5 and 6. Any of the foregoing may have one, two, three, four or more spiral grooves along the surface to increase the surface area of the equilibration member.

Where the equilibration member is in the form of a hemisphere, a hemiellipsoid, a hemiovoid, or a domed frustum, the equilibration member may be formed against or as part of the lower portion of the chamber. See, e.g., FIGS. 6E-6G.

In one embodiment the equilibration member is substantially spheroidal, ellipsoidal or ovoidal. In such an embodiment the equilibration member may be a sphere, ellipsoid, or ovoid.

In an embodiment the equilibration member is substantially spheroidal.

In an embodiment the equilibration member is substantially ellipsoidal.

In an embodiment the equilibration member is substantially ovoidal.

The equilibration member may occupy a volume that is greater than 50%, 60%, 70%, 75%, 80%, 85%, 90%, or 95% of the interior volume of the chamber (e.g., from 50% to 70%, from 60% to 80%, from 70% to 90%, from 80% to 95%, or from 90% to 95%). In an embodiment, where the chamber is substantially a virtual right cylinder (VRC), the equilibration member may occupy greater than about 50%, 60%, 70%, 75%, 80%, 85%, 90%, or 95% of the interior volume of the chamber. In another embodiment, where the chamber is substantially a VRC, and the equilibration member is spheroidal, the volume of the equilibration member is less than about 70% of the interior volume of the chamber (e.g., less than 65%, 60%, 50%, 40% or 30% of the interior volume of the equilibration chamber).

As discussed above, the equilibration member may have a surface over which the liquid can form a continuous film over all of its surface area, or the liquid can form a continuous film over greater than 50%, 60%, 70%, 80%, 90% or 95% of its surface area (e.g., flow is not impeded by the shape of the design). In such an embodiment the equilibration member may be wettable by the liquid. In an embodiment, where the liquid subject to measurement is an aqueous liquid, the equilibration member is hydrophilic. In such an embodiment, the static contact angle of the equilibration member with water can be less than any of 90°, 80°, 70°, 60°, 50°, 40°, 30°, 20°, or 10° measured by a goniometer (e.g., sessile drop analysis using an Attension® Theta Flex from Bolin Scientific per manufacturer's procedure) at 22° C. in air. Alternatively, the static contact angle of the equilibration member with water can be less than 60° or less than 500 (e.g., less than 40°, less than 30°, less than 20°, or less than 10°) measured by a goniometer (e.g., sessile drop analysis using an Attension® Theta Flex from Bolin Scientific per manufacturer's procedure) at 22° C. in air.

The equilibration members themselves may be made from a variety of materials and may be, for example, hollow, solid or made of a shell filled with another material. Where a shell type structure is used, the equilibration members may be filled with a foam or foam-like material (e.g., a polyurethane foam) having a closed or open cell structure. Where equilibration members are hollow, they are designed to be totally sealed, or sealed sufficiently so that only an insubstantial amount of gas or liquid can enter the member's interior space(s). For example, a hollow equilibration member may have a small hole (e.g., pin hole) to prevent pressure differences with the gas and/or liquid within the chamber.

### 2. Chambers

The chamber of the equilibration apparatus can serve a variety of purposes including positioning and supporting the equilibration member and the gas and liquid inlets and outlets. The chamber may have any suitable shape provided it does not interfere with the passage of gas through the chamber or the formation or movement of the falling film of liquid introduced into the chamber as it is drawn downward by gravity over the surface of the equilibration member. The chamber comprises an outer wall having a thickness t, with the wall defining the interior surface of the chamber, the exterior surface of the chamber, and space within the chamber. In various embodiments the chamber wall is disposed substantially symmetrically about a central axis and may have a cylindrical, spheroidal, ellipsoidal or ovoidal shape. Where the chamber is spheroidal, it may be a sphere, or it may have a prolate or oblate spherical shape. Regardless of exact shape of the chamber, for minimization of response dead time and rapidity of the response time, chamber headspace will be closer to optimization when the chamber's interior surface is substantially parallel to, or substantially follows the contour of, the equilibration member's outer surface.

As discussed above, although the chamber may have a variety of shapes, where the chamber substantially conforms to the shape of the equilibration member, the headspace (volume) within the chamber surrounding the equilibration member and the film of falling liquid is minimized. Minimizing the space within the chamber around the equilibration member permits the equilibrator to more rapidly respond to changes in the gas content of the liquid in the falling film. The improved response time is a function of, among other things, the more rapid turnover of the gas within the chamber at any given carrier gas flow rate and the smaller volume of gas in the chamber with which the incoming carrier gas will be mixed. The response time can also be improved by limiting areas within the chamber that may form eddies or interfere with the laminar flow of carrier gas from the gas inlet to the gas outlet. Accordingly, in embodiments, both the equilibration member and the chamber may have a shape that is spheroidal, ellipsoidal, or ovoidal.

In an embodiment, the chamber is in the form of a VRC. Where the chamber has the overall shape of a VRC, the equilibration member housed within it may be of any shape discussed above including spheroidal. Where the chamber is in the form of a VRC, and it is desirable to minimize the space around the equilibration member, the equilibration member may have an ellipsoidal, an ovoidal, or a prolate or oblate spheroidal shape.

Regardless of its shape, the chamber, the exterior surface of the chamber, the chamber wall, the equilibration member within the chamber, and the space within the chamber may be conceptually divided into an upper portion and a lower portion. The upper portion is defined as the section above the level of the equilibration member's bisecting plane and the lower portion as the section below that bisecting plane when the equilibration member is located within the chamber in position for the apparatus to operate such that its axis of rotation and the central axis of the chamber coincide or substantially coincide.

The chamber may be formed with a chamber wall section that is removably-resealable to permit access to the interior of the chamber. The section may be of sufficient size to permit access for monitoring, cleaning and maintenance, or even removal of the equilibration member for inspection, cleaning and/or replacement. The seal may be any suitable type, including those formed by O-rings, gaskets, snap-fit, compression or frustoconical sections (e.g., with a seat and threaded sections), or combinations of any thereof. The section of the chamber wall that is removably-resealable may be located in the lower portion of the chamber. Alternatively, the section that is removably-resealable may be located in the upper portion of the chamber. Where the chamber is in the form of a VRC, or substantially in the form of a VRC, the removably-resealable section may constitute all or part of the planar upper surface of the cylinder. In an embodiment, the removably-resealable section comprises the upper planar surface of the VRC and a seal at or proximate to its circumference that engages all or part of the cylindrical wall of the cylinder. In such an embodiment the vertical wall of the VRC may comprise one or more ridges to retain the removably-sealable upper section and/or a seal or a sealing surface that engages the upper portion.

The interior volume of a chamber may be varied over a substantial range, for example from about 1 to about 25 liters (e.g., from about 1 to about 4, from about 1 to about 8, from about 9 to about 16, from about 12 to about 25, from about 16 to about 20, from about 18 to about 25, or from about 20 to about 25).

| | | | | | Approx- |
|---|---|---|---|---|---|
| | | | | | imate |
| VRC | | | | | Ratio of |
| chamber | | | | Approx- | the |
| and | | | | imate | Chamber |
| Spheroidal | Cham- | Approx- | Equili- | Equili- | Volume to |
| Equili- | ber | imate | bration | bration | Equili- |
| brator | Diam- | Chamber | Member | Member | bration |
| Combi- | eter | Volume | Diameter | Volume | Member |
| nation | (cm) | (cm³) | (cm) | (cm³) | Volume |
| #1 | 11-12 | 950-1360 | 10 | 524 | 1.8-2.9 |
| #2 | 13-14 | 1600-2440 | 12 | 904 | 1.8-2.9 |
| #3 | 15-16 | 2600-3900 | 14 | 1437 | 1.8-2.9 |
| #4 | 17-18 | 3900-5800 | 16 | 2145 | 1.8-2.9 |
| #5 | 19-20 | 5500-8250 | 18 | 3053 | 1.8-2.9 |
| #6 | 21-22 | 7500-11300 | 20 | 4189 | 1.8-2.9 |
| #7 | 23-24 | 10000-15000 | 22 | 5575 | 1.8-2.9 |
| #8 | 25-26 | 13000-19550 | 24 | 7238 | 1.8-2.9 |
| #9 | 27-28 | 16550-24850 | 26 | 9202 | 1.8-2.9 |
| #10 | 29-30 | 21500-31000 | 28 | 11494 | 1.8-2.9 |
| #11 | 31-32 | 25450-38200 | 30 | 14137 | 1.8-2.9 |
| #12 | 33-34 | 30900-46300 | 32 | 17157 | 1.8-2.9 |

Using a chamber that has a shape that substantially matches the contoured shape of the equilibration member, the headspace in the chamber can be reduced. For example, a spheroidal chamber and spheroidal equilibration member combination can have chamber volume:equilibration volume ratio from about 3.3 to 2.0 with an equilibration member to chamber headspace ratio from about 0.3 to 1.0 (headspace volume divided by equilibration member volume).

### 3. Positioning of the Equilibration Member and the Location of the Inlets and Outlets

The equilibration member may be positioned and held in place within the chamber in a number of different ways, including those that are permanent (affixing the equilibration member to the interior of the chamber in a non-removable manner) or non-permanent (holding the equilibration member in place by contact with the chamber interior or supports within the chamber interior (e.g., annular supports, pedestals, etc.).

Examples of permanent ways of affixing the equilibration member to the interior of the chamber include the use of adhesives or fusing the equilibration member to the chamber at one or more points.

Non-permanent methods of positioning and holding the equilibration member in place permit the removal of the equilibration member from the chamber for cleaning and/or servicing the apparatus.

In an embodiment, the equilibration member is held in place in a non-permanent manner by gravity and is directly removable from the chamber once all or part of a section of the chamber wall that is sufficient in size to extract the equilibration member is removed. Among the non-permanent structures that may be used to retain the equilibration member in place are studs and/or rings on the interior surface of the chamber that position and hold the equilibration member in place during operation by contacting it. Alternatively, studs and/or rings may be on the surface of the equilibration member and hold the member in place by contacting the interior surface of the chamber. Another alternative is the use of a combination of studs and/or rings attached to the chamber and equilibration member. The use of non-permanent methods of positioning the equilibration member permits the equilibration member to be removed (e.g., lifted) out of the chamber for cleaning and maintenance of the member and/or chamber once a removably-resealable chamber wall section is disengaged and the chamber is opened.

The equilibration member may also be affixed to the chamber using non-permanent connections such as screws, clamps, latches, magnets and the like that can be removed or uncoupled to free the equilibration member and permit its removal from the chamber once a removably-resealable chamber wall section is disengaged and the chamber is opened.

In an embodiment, a cylindrical pedestal is placed vertically beneath the equilibration member with the axis of rotation of the cylindrical member, the equilibration member, and the central axis of the equilibration chamber all substantially aligned. The pedestal, which may be permanently or non-permanently affixed to the equilibration member, positions the equilibration member properly for generation of falling liquid film and also serves as an additional falling film surface as liquid transitions from the equilibration member and flows over the pedestal surface before draining out of the container. Where the pedestal is solid or does not readily permit gas to exchange with any space within the pedestal, the volume of the pedestal positioning member also reduces the amount of headspace volume inside the equilibrium chamber.

In an embodiment, the equilibration member is positioned within the chamber by a ring, annular projection, or concave section formed in the lower portion of the chamber. In such an embodiment, the equilibration member may be made of a magnetically susceptible material or comprise a magnet or magnetically susceptible material, such that the equilibration member may be magnetically engaged to the interior surface of the chamber by a magnet located (positioned) on or in the chamber wall. The equilibration member may also be magnetically engaged in a position proximate to, but not in direct contact with, the chamber wall (e.g., the lower portion of the chamber wall) where it is supported by studs or an annular element (ring). Such an embodiment is shown in FIG. 3, where the chamber is a VRC or substantially a VCR, and a spherical equilibration member is held against an annular element that is in contact with the planar or substantially planar lower interior surface of the VRC.

In an embodiment the equilibration member is mounted inside a chamber using a series of stand-off posts alone or in combination with an annular element.

In an embodiment the equilibration member is positioned inside the equilibration chamber by floating on a surface of the liquid (e.g., water) that accumulates at the bottom of the equilibration chamber prior to draining. In this embodiment, a spherical equilibration member can either rotate freely or remain relatively stationary depending on the attack angle and force of the water introduced into the chamber and onto the member through an inlet port (e.g., the liquid in the chamber acts as a hydrodynamic bearing). In such an embodiment, the equilibration member can be kept approximately centered in the chamber by the use of small posts or ribs (e.g., either parallel to or perpendicular to the central axis) on the chamber's inside surface.

In an embodiment, the equilibration member may be suspended from the upper portion of the chamber. In one such embodiment, the equilibration member is suspended by a flexible material (e.g., a strand of wire, string, plastic, fiberglass, rubber etc.) from the upper portion of the chamber at or near the point where the central axis passes through the chamber wall (e.g., at or near the center of the lid). Equilibration members suspended from the upper portion of the chamber can act like a pendulum and have the tendency to stay centered under the liquid entering the chamber from a centrally located liquid inlet when the chamber is tilted.

Equilibration members are generally positioned in the chamber such that there is a gap between the equilibration member and the chamber wall. The gap permits a liquid (e.g., water) introduced into the upper part of the chamber that runs over the equilibration member to reach the lower portion of the chamber unimpeded. At the same time, air or a carrier gas introduced into the lower part of the chamber via the gas inlet can freely move to the upper portion of the chamber through the gap. The gap is generally distributed uniformly around the equilibration member but there does not have to be a completely uniform gap and the equilibration member may even contact the chamber wall at one or more points. While the size of the gap between the chamber wall and the equilibration member may vary, a gap in the range of 0.1 cm to 2.5 cm (0.1-0.5, 0.5-1.0, 1.0-1.5, 1.0-2.0, 1.5-2.5, or 2.0-2.5) will generally be sufficient to permit passage of the gas and the liquid.

In an embodiment, the chamber is a VRC having an interior volume of from about 1.6 liters to 25 liters and a diameter of about 6.5 cm to about 33 cm. In such an embodiment, a spherical equilibration member having a diameter that is from about 0.2 cm to about 5 cm (e.g., about 0.2 cm to about 1.0 cm, about 0.2 cm to about 2.0 cm, about 2.0 cm to about 4.0 cm, or about 2.5 cm to about 5 cm) less than the inner diameter of the chamber may be employed. Accordingly, where there is a difference in diameter of about 0.2 cm to about 5 cm and the equilibration member's axis of rotation and the central axis of the chamber are aligned, there will be a uniform gap of from about 0.1 cm to about 2.5 cm (e.g., about 0.1 to about 0.5 cm, about 0.5 to about 1.0 cm, about 1.0 to about 2.0, or about 2.0 to about 2.5 cm) between the equilibration member and the chamber wall at the equator of the equilibration member or the location of the equilibration member with the greatest diameter or radius.

In another embodiment, the chamber is spherical and has a volume of from about 1.0-18.0 liters (about 12.7 cm to about 33.0 cm in diameter), and the equilibration member is spherical and has a diameter that is less than the inner diameter of the chamber's interior by about 0.2 cm to about 8 cm. Accordingly, when the equilibration member's axis of rotation and the central axis of the chamber are aligned, there will be a uniform gap of from about 0.1 cm to about 4 cm (e.g., about 0.5 to about 1.0 cm, about 1.0 to about 2.0, or about 2.0 to about 4 cm) between the equilibration member and the chamber wall.

The liquid inlet(s) may be positioned in the upper portion of the chamber such that liquid introduced via the liquid inlet(s) can form a film over greater than 50%, 60%, 70%, 80%, 90%, or 95% of its surface area as the liquid is drawn downward over the equilibration member by gravity. Liquid inlets may include liquid inlet nozzle(s) that direct the stream of incoming liquid at the equilibration member. The liquid stream may be introduced at a relatively slow rate such that gravity will substantially control the location where the liquid will strike the equilibration member. Alternatively, the liquid may be introduced as a stream that can be directed at the equilibration member by the liquid inlet (nozzle). In such embodiments, the liquid stream may be directed such that it will impact the surface at an angle that is perpendicular to the surface of the equilibration member at the point of impact.

In an embodiment, the introduction of liquid may be accomplished using a single liquid inlet (e.g., a liquid inlet nozzle) located at the point where the central axis of the chamber intersects the upper portion of the chamber wall. For example, where the chamber is a VRC or a spheroid, a single liquid inlet may be located at the center of the upper planar surface of the VRC or at the top of the spheroid respectively. The use of a single inlet located where the central axis of the chamber intersects the upper portion of the chamber wall permits the equilibrator to be operated when the central axis of the equilibrator (and the axis of rotation of the equilibration member) are displaced from about 0° to about 150 or more from the vertical (e.g., the equilibrator may be tilted from about 0° to about 100 or from about 0° to about 15°) without disruption of its operation.

In other embodiments more than one liquid inlet (e.g., nozzle) may be located in the upper portion of the chamber such that water from one, two, three or more inlets is directed at the surface of the equilibration member. In one embodiment the inlets are spaced around (e.g., equidistant from) the point where the central axis of the chamber intersects the upper section of the chamber surface. Such embodiments include the placement of the liquid inlets at the corners of regular polygons (e.g., triangle, square, pentagon, hexagon, heptagon, or octagon) centered at the point where the central axis of the chamber intersects the upper section of the chamber surface. The liquid inlet(s), regardless of how they are arranged, may be placed in a portion of the chamber wall that is removably-resealable, or in a portion of the chamber wall that is not removably-resealable with the portion of the chamber that retains the equilibration member. For example, where the chamber is substantially in the form of a VRC, all or part of the substantially planar upper surface of the VRC may act as a “lid” for the remainder of the chamber.

As the equilibrators are of a design that is substantially symmetrical about the central axis of the chamber, the equilibrator can be operated when the central axis is displaced from the vertical in any direction. As indicated above, the equilibrators may be operated when the central axis is displaced from about 0° to about 150 from the vertical. The use of higher liquid flow rates and/or liquid inlets with nozzles that direct liquid at the equilibration member increases the angle at which the equilibrator may be operated. In one embodiment the nozzles may extend into the chamber terminating proximate to the equilibration member such that they direct the incoming liquid at the equilibration member at an angle that is substantially normal to the equilibration member's surface at the point where the liquid stream contacts the equilibration member. In some embodiments the equilibrator may be operated when the central axis is displaced (the equilibrator is tipped) up to about 20°, 25° or 30° from the vertical. The ability of the equilibrator to operate when tipped permits its use on, for example, floating platforms where waves may rock the equilibrator.

Liquid inlets and tubing bringing liquids to the inlets will typically have an inner diameter greater than 2 mm, for example in the range of about 2.0 to about 14.0 mm (e.g., 2.0-4.0, 2.0-6.0, 4.0-8.0, 6.0-10.0, 8.0-14.0. or 10-14 mm). Liquid inlets may terminate at or be in the form of a nozzle that extends into the chamber to direct the stream of incoming liquid at the equilibration member. Nozzles, when present, will be in the same size range as the tubing bringing liquid to the inlets, namely about 2.0 to about 14.0 mm (e.g., 2.0-4.0, 2.0-6.0, 4.0-8.0, 6.0-10.0, 8.0-14.0. or 10-14 mm). In an embodiment where the chamber is in the form of a VRC, the liquid inlets are placed on the substantially planar upper portion of the chamber and may be distributed as described above—with regard to the central axis.

One or more liquid outlets are located in the lower portion of the chamber and positioned to permit outflow of some or all of the liquid that collects in the lower portion of the chamber by gravity. To prevent the atmospheric gas in which the equilibrator is operated from entering the equilibrator, the liquid outlet 2 may be fitted with a trap or one-way drain (e.g., a p-trap or duck billed one-way drain). Where the chamber is in the form of a VRC, the liquid outlet(s) may be in the cylindrical side wall of the chamber and/or in the substantially planar lower surface of the chamber. Where the chamber is spheroidal, ovoidal, or ellipsoidal (see FIG. 4 and FIG. 6 at a-d), a single liquid outlet may be located at the point where the central axis of the chamber intersects the lower portion of the equilibrator chamber. In an embodiment, the chamber has the overall shape of a VRC, with the lower surface of the chamber modified either to a convex or conical shape (see, e.g., FIG. 6 at o) and/or to accommodate channels that direct liquids that are drawn to the bottom of the chamber by gravity toward the one or more liquid outlets 2 in the convex or conical surface and/or in the channels. Under proper flow conditions, a liquid outlet located in the substantially planar lower surface of an equilibrator in the form of a VRC minimizes sediment/debris buildup. Minimization of sediment/debris buildup can be enhanced by making the chamber floor slightly convex or conical to function like a funnel (see FIG. 4 and FIG. 6 at a-d and o).

Vortexing and unwanted gas loss from the equilibrator can be avoided by calibrating (adjusting) the outlet diameter with liquid flow rate such that during steady state operation a pool of liquid (e.g., carrier liquid, test liquid or calibration liquid) having a critical height above the liquid outlet (i.e., height as measured to the liquid's free-surface, or its “depth”) sufficient to prevent vortexing is formed. This can be achieved by using liquid outlets with different fixed orifices, or by using a valve to control flow from the liquid outlet. Anti-vortexing devices can also be positioned in relation to the liquid outlet to preclude vortexing by physically influencing liquid flow patterns. In equilibrator embodiments where the chamber has a conical or convex lower portion (see, e.g., FIG. 4), liquid outlet 2 can be centered at or near the lowest portion of the equilibrator chamber (conical or flat floor) and the equilibrator member is positioned/supported with an annular device (“as” in FIG. 4) containing slots, louvers, etc. that disrupt vortexing. In such an embodiment, the annular support 1) locates the equilibration member inside the chamber and 2) simultaneously functions as an anti-vortex device.

Liquid outlets and the tubing carrying liquid away from the outlet will typically have an inner diameter of a similar size to the liquid inlet; however, where the liquid inlet is typically under pump pressure and the outlet passively drains liquid under the force of gravity, the diameters may deviate somewhat. More specifically, the liquid outlet will generally have an inner diameter greater than 2 mm, for example in the range of about 2.0 to about 20.0 mm (e.g., 2.0-4.0, 2.0-6.0, 4.0-8.0, 6.0-10.0, 8.0-14.0, 10-14, or 14-20 mm). During operation of the equilibrator the liquid outlet should not permit air to enter the chamber by allowing liquid to drain away too quickly, and at the same time liquid should not flood/overfill the chamber. Accordingly, the liquid outlet may be sized to maintain at least some liquid in the lower portion of the chamber. The cross-sectional area of the liquid outlet, or a portion of the tubing attached to it may be adjustable and set to accommodate specific liquid out-flow rates by use of a valve or clamp that compresses/constricts the liquid outlet and/or the tubing attached to the outlet. Alternatively, a U-shaped liquid trap, a valve, a one-way flow valve (e.g., a check valve, flapper valve or feather valve) installed in the tubing that carries liquid away from the equilibrator may be utilized to prevent air from entering the chamber through the liquid outlet.

Regardless of the shape of the chamber, placement of at least one of the one or more liquid outlets at or near the lowest point of the chamber (as determined when the central axis of the chamber is vertical) permits the clearance of solid/semisolid materials (e.g., sediment) from the chamber, which extends the time between cleanings necessary to maintain proper operation (gas equilibration). Where the equilibration member is positioned in the chamber by a ring, annular projection, or concave section formed in the lower portion of the chamber, any of those elements may be provided with channels, grooves or gaps so that liquid can reach the liquid outlet. The ring or annular projection may be of any suitable dimension. In an embodiment the ring will be formed from a hoop of a material (e.g., tubing) having a circular cross-section with a diameter of about 8 to about 30 mm. Annular projections built into the wall of the chamber may be of similar size and shape to rings used to support the equilibration member (e.g., about 0.7 to about 2 cm in diameter/height). As with rings, annular projections built into the chamber wall used to support the equilibration member are provided with gaps or openings to permit the flow of liquids to and from the space below the equilibration member as needed, for example where the liquid outlet may be located. The equilibration chamber may be provided with external supports (e.g., external legs or a pedestal) to provide stability when placed on a horizontal surface and/or where the liquid outlet is located low enough on the chamber (e.g., on the bottom of a VRC chamber) that it would interfere with stable placement of the apparatus on a horizontal support surface.

One or more gas inlets are located in the wall of the lower portion of the chamber. Gas inlets are generally placed in the wall of the lower portion of the chamber at a point above the level of the liquid outlet(s). Placement in that manner, however, is not required provided that any mechanism used to introduce gas into the chamber provides sufficient pressure to prevent liquid in the chamber from backing up into the gas inlet(s) or the tube(s) supplying gas to the gas inlet(s).

Accordingly, in an embodiment, the gas inlet(s) may be placed in the wall of the lower portion of the chamber at a point above the level of the liquid outlet(s) as determined when the central axis of the chamber is vertical. In another embodiment, the gas inlet(s) may be placed in the wall of the lower portion of the chamber above the level at which liquid can accumulate in the chamber as determined when the central axis of the chamber is 10°, 12°, 15°, 20°, 25° or 30° from the vertical, taking into consideration the location of the liquid outlet(s). In other embodiments the gas inlets are placed in the wall of the lower portion of the chamber at or below the level of the liquid outlet(s) as determined when the central axis of the chamber is vertical. In such an embodiment gas entering the chamber will bubble through the liquid as it enters.

Gas inlet(s) may terminate in a nozzle that diffuses the gas as it enters the chamber or directs the gas entering the chamber in a specific direction. A combination of nozzles that diffuse gas or direct it in one or more directions may be employed. In an embodiment, where gas enters the chamber in a diffuse undirected fashion, it will cause turbulence in the gas in the lower portion of the chamber that may assist in the equilibration of the gases present in the liquid with the gas phase. In another embodiment, nozzles may direct a stream of gas entering the chamber toward the central axis of the chamber. In other embodiments, nozzles may direct gas entering the chamber away from the central axis of the chamber. For example, nozzles may direct gas entering the chamber along the interior surface of the chamber (parallel to the wall) at the point where the nozzle is located, thereby directing the gas in the lower portion of the chamber to circulate around the central axis.

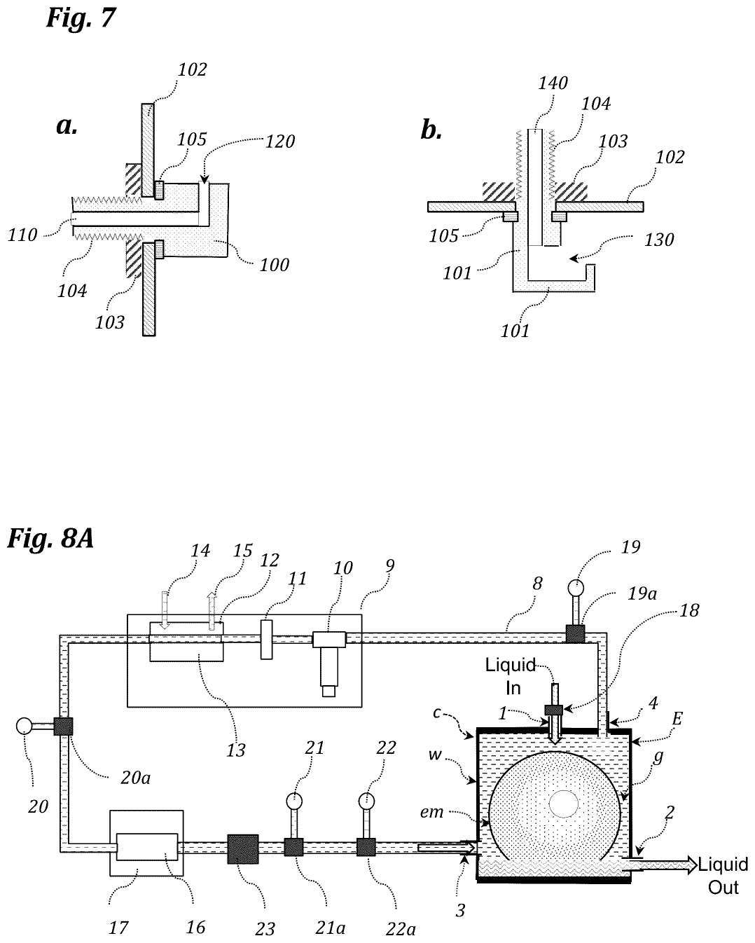

One or more gas outlet(s) are located in the upper portion of the chamber's wall and may be provided with a shield to prevent droplets of liquid that splash in their direction from entering the gas outlet (see FIGS. 7A and 7B). The gas outlets are positioned to avoid the intake of liquid and, accordingly, may be located in the chamber wall above the level of the liquid inlet(s). Where the liquid inlet(s) comprise a nozzle that extends into the chamber, the gas outlets may be located above the level where the liquid is discharged from the nozzle. Where the chamber is in, or substantially in, the form of a VRC, the gas outlets may be positioned on the substantially planar upper surface along with the liquid inlet(s). In an embodiment, the gas inlet(s) are located in a portion of the chamber wall that is removably-resealable with the portion of the chamber that retains the equilibration member. In such an embodiment, the removably-resealable portion of the chamber wall with the gas outlets may also contain one or more of the liquid inlets. Gas outlets also may be located in the upper portion of the chamber's wall that is not removably-resealable. In an embodiment, where the chamber is substantially in the form of a VRC, one or more of the liquid inlets and/or one or more of the gas outlets may be located in the substantially planar upper surface of the VRC, which acts as a “lid” for the remainder of the chamber.

The inner diameter of the gas inlets and outlets, and of the tubing connected to them, may be of any suitable size to accommodate the flow of gas to and from the chamber. In an embodiment, the outside diameter of the gas inlets and outlets, and of the tubing connected to them, may be up to about 8 mm (e.g., up to about 6 mm or in the range of from about 4 to about 8 mm) with a wall thickness of, for example, about 0.5 mm or less, giving an inside diameter up to about 7 mm (e.g., from about 3 mm to about 7 mm). In other embodiments, the gas inlet(s), the gas outlet(s) and the tubing connected to them, each have an inner diameter selected independently from a range selected from: 1-12.5, 1-2, 2-4, 2-6, 2-8, 4-6, 4-8, 4-12.5, 6-10, 6-12.5 and 8-12.5 mm.

### 4. Temperature Regulation

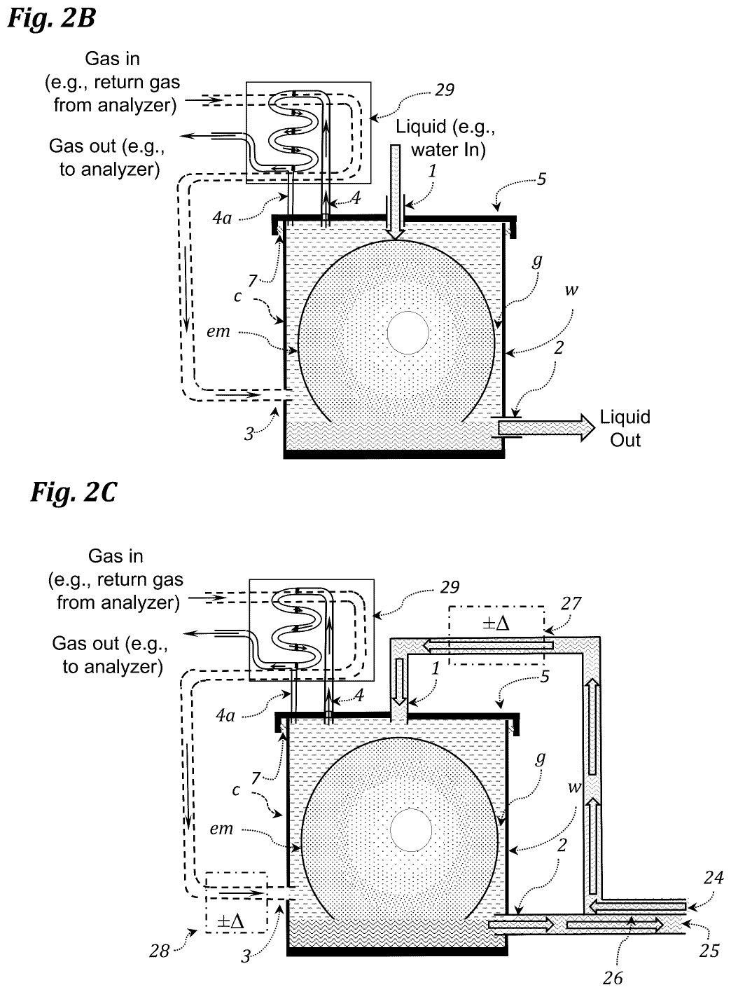

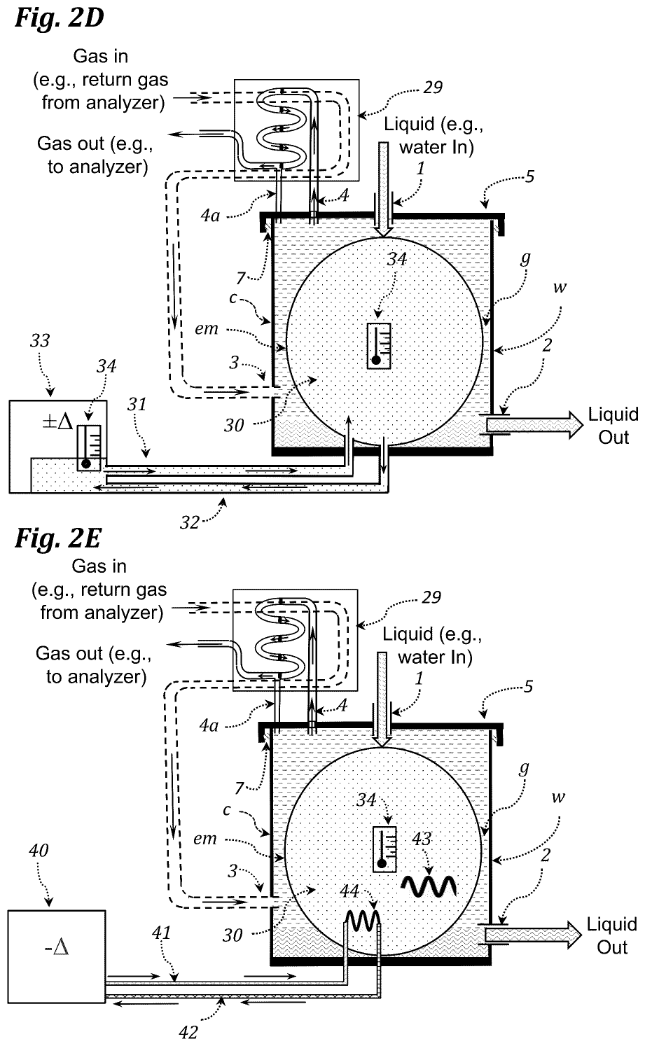

The equilibrators described herein may include one or more mechanisms for regulating the operating temperature of the equilibrators described herein (i.e., the temperature of the equilibration reaction). Temperature regulation includes increasing the temperature at which the equilibrator operates in order to increase the rate of equilibration between one or more gases or volatile substances dissolved in a liquid and a carrier gas. The mechanisms include but are not limited to: (i) a liquid inlet temperature control element 27 for heating the liquid prior to its entering into the chamber: (ii) heating and/or cooling mechanisms for the equilibration member, and/or (iii) a gas inlet heating/cooling element 28 for heating the gas stream (carrier gas) entering the equilibrator via inlet 3. Any of those mechanisms may employ heat sources including, but not limited to, electrical resistance heating elements (e.g., metal, ceramic and semiconductor, composite heating elements, positive and negative temperature coefficient thick film heaters), radiant heating, induction heating, thermoelectric heating/cooling (e.g., using a Peltier element), microwave heating or a combination thereof. Cooling may be provided by refrigeration units, Peltier elements, cooling fluid (e.g., cooling water) and the like.

Where equilibrators are equipped with a liquid inlet temperature control element (e.g., item 27 in FIG. 2C), it may comprise electrical resistance heating elements (e.g., metal, ceramic and semiconductor, composite heating elements, positive and negative temperature coefficient thick film heaters, etc.), radiant heating elements (e.g., infrared emitting lamps/heat lamps or incandescent lamps), induction heating, or microwave emitters (e.g., a klystron or a magnetron that may be tunable) and can be positioned to heat the incoming fluid, the equilibration member, and/or the carrier gas stream. The heating may be direct (e.g., as in water heated by microwaves) or indirect (e.g., by heating the tubing or nozzle(s) through which the gas or fluid flows). Cooling may be provided by refrigeration units, Peltier elements, cooling fluid (e.g., cooling water) and the like. Liquid inlet temperature control element 27 may comprise one or more electrical resistance heating elements and/or one or more microwave emitters. Alternatively, element 27 may comprise a heat-transfer fluid that is i) heated by, for example, electrical resistance heating elements within 27, or ii) recirculated between element 27 and an external heat source. In the case of a recirculating heat-transfer fluid, electrical heating or either active or passive solar heating could be utilized as an external contributing heat source.

Providing heat and/or cooling to the equilibration member, and particularly providing heat and/or cooling directly or indirectly to the inside surface of the equilibration member offers the advantage of providing a high surface area for heat transfer while leaving the falling film gas exchange surface of the member uninterrupted. In equilibrator designs where the equilibration member, particularly the inside surface of the equilibration member(s) is heated, heat may be applied to the member directly by electrical resistance heating elements, radiant heating elements, induction heating elements, or microwave emitters. Alternatively, a thermostatically controlled heating and/or cooling element can be positioned inside an equilibrator member that contains a heat-transfer fluid that is enclosed inside the equilibration member(s) (see 30 in FIG. 2E). The thermostatically controlled heating/cooling element may comprise, for example, both a heating element 43 (e.g., an electrical resistance heater) and a cooling element 44 (e.g., refrigerant cooled element that removes heat to the exterior of the equilibrator). Heating or cooling the heat-transfer fluid indirectly provides heating or cooling to the equilibration member. Expansion and contraction of the heat exchange fluid can be compensated for by an encapsulated gas filled bladder that can be compressed or by an expansion tank external to the equilibration member (not shown).

Another mechanism for indirectly heating or cooling the equilibration member is depicted in FIG. 2D, where there is an equilibration member filled with a heat-exchange fluid 30 that provides temperature regulation of the equilibration member by circulating or recirculating the heat-exchange fluid with an external heating and/or cooling unit 33. Unit 33 may be, for example, a temperature-controlled bath of heat-exchange fluid (e.g., an aqueous ethylene glycol or aqueous propylene glycol mix). In the case of a recirculating heat-transfer fluid, active (e.g., using a solar collector panel) or passive solar heating could also be used as a contributing external heat source.

Where the equilibration member is heated/cooled, particularly when heated directly or indirectly from its inside, the equilibration member is advantageously comprised of a material with suitable heat conductivity (e.g., from 10 to 200 W⁻¹K⁻¹ or more than 200 W⁻¹K⁻¹. The equilibration may be prepared from a material selected to have a thermal conductivity from about 10 to about 50 W⁻¹K⁻¹ or from about 50 to about 100 W⁻¹K⁻¹. The equilibration member may be prepared from a material selected to have thermal conductivities from 100 to 200 W⁻¹K⁻¹ or greater than 200 W⁻¹K⁻¹ (e.g., copper with a thermal conductivity greater than 350 W⁻¹K⁻¹ that is optionally plated to prevent corrosion). Where copper or its alloys are employed, it may be plated to prevent corrosion; however, in certain environments (e.g., marine and other aqueous salt environments) the inherent antifouling/anticorrosion behavior of copper and its alloys may be effective and plating is not required.

The heating/cooling of gas (e.g., an inert carrier gas or air) introduced into the equilibrator through the gas inlet line 3 may be accomplished using a gas inlet heating/cooling element 28. As indicated above, heat may be supplied using heating elements, radiant heating elements, induction heating elements, or microwave emitters, etc., that can be positioned to heat the incoming carrier gas stream. Because most gases have a lower heat capacity than most liquids subject to analysis (test liquids), heating and cooling of gases as a mechanism for controlling the temperature of the equilibration reaction will typically be conducted along with one or more other mechanisms of temperature control.

Regardless of the mechanism used to control the temperature of the equilibration reaction, the process can be made more efficient by partially or completely equilibrating the temperature of the incoming liquid or carrier gas with either the liquid or gas leaving the equilibrator via outlets 2 or 4. By way of example, in those instances where the incoming liquid entering the system from a liquid supply 24 requires heating, the liquid exiting the equilibrator via liquid outlet 2 can be directed to a heat exchanger where it is used to warm incoming test liquid. In some instances, the heat exchange will be via a counter current heat exchanger such as that shown schematically as item 26 in FIG. 2C. The heat exchanger (e.g., counter current heat exchanger) may be separate from or integrated into the liquid inlet temperature control element 27. Similarly, where incoming liquid requires cooling, it can first be partially cooled by liquid exiting the equilibrator using a heat exchanger. Where the equilibrator is operated without constraints on energy consumption, the improvements in efficiency may not be warranted; however, where the equilibrator is operated remotely and energy for its operation may be a constraint, the improved efficiency may be warranted. The use of thermal insulation materials on the outside of the equilibration chamber and lines containing liquid and or gas streams may make temperature control more efficient and minimize unintended heat gain and/or loss from the equilibrator.

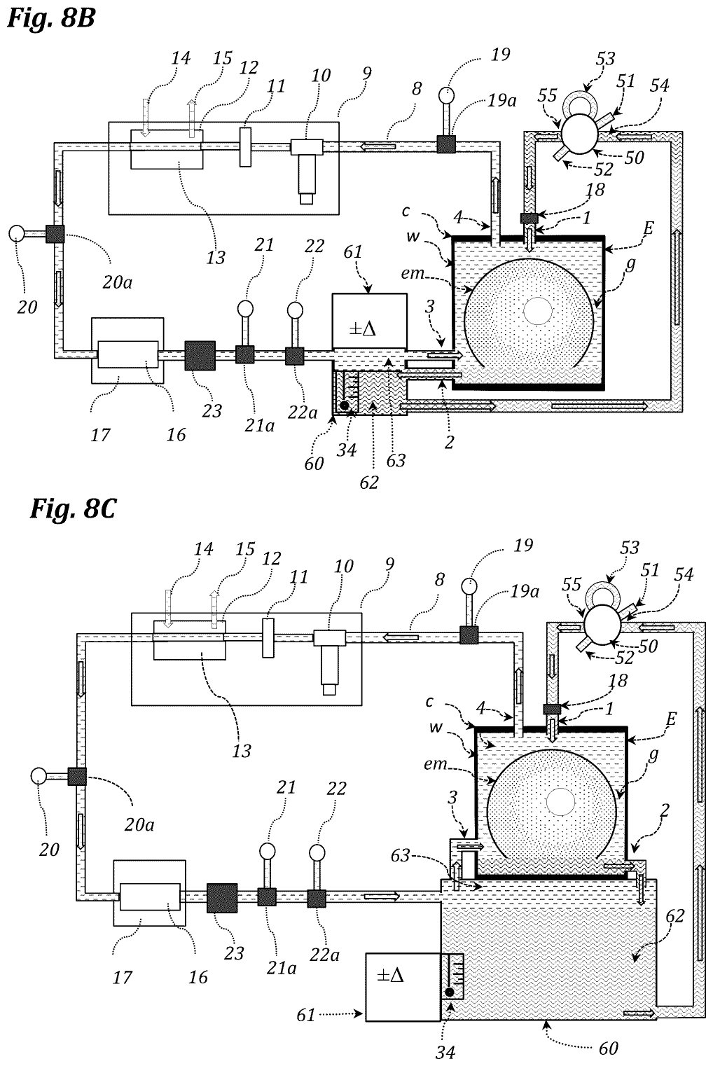

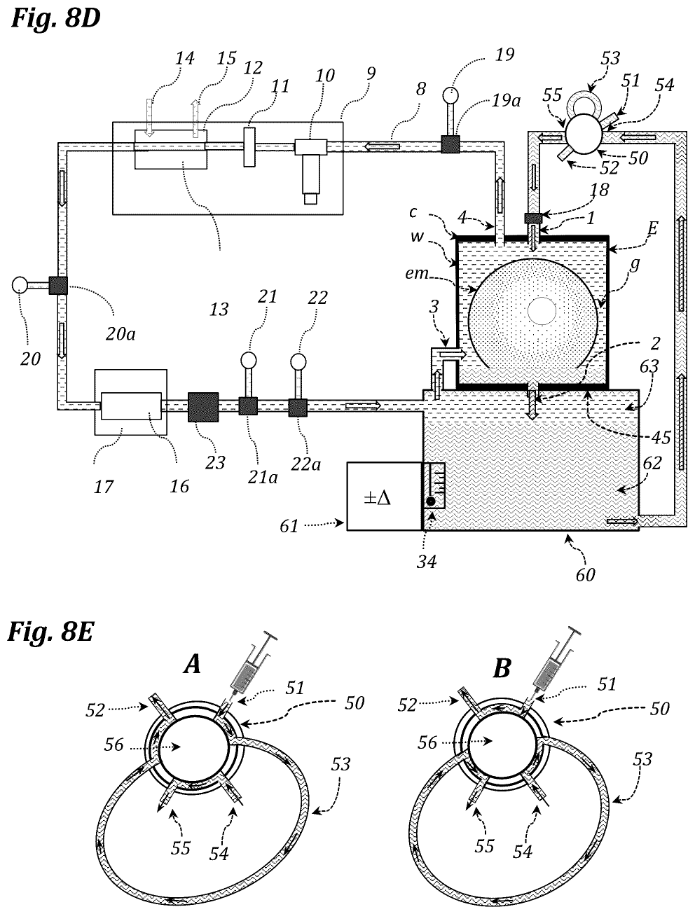

Gas exiting the equilibrator by gas outlet line 4 may contain a substantial amount of the liquid vapor in addition to the dissolved gases and volatile substances that are to be measured. The gas stream may optionally be heated (e.g., by electrical resistance heating or thermoelectric/Peltier heating) in order to prevent condensation of volatile substances and/or components of the liquid (e.g., water vapor) during analysis. Optionally, the gas stream may be cooled to condense components of the gas stream (e.g., water vapor), while allowing dissolved gases and other volatile substances to remain in the gas stream subject to analysis. Cooling may be accomplished by contacting the gas stream with a surface (e.g., the interior of a tube) cooled with a Peltier thermoelectric cooler/heater as exemplified by element 29 in FIGS. 2B to 2D. Heat extracted from the gas stream exiting the equilibrator chamber can be lost by the heated side of the thermoelectric cooler/heater by convection, by radiation, by transfer to the liquid entering the chamber as part of liquid inlet temperature control element 27, or by transfer to the gas being returned to the equilibrator following analysis as shown in FIGS. 2B and 2C. Condensate formed upon cooling the gas may be returned to the equilibrator chamber (e.g., by condensate return line 4a), added to the liquid exiting the chamber by liquid outlet 2, or otherwise disposed of (e.g., returned to the source of the test liquid). Systems to cool and condense vaporized test liquid (or carrier liquid), including those with thermoelectric cooling (e.g., diagramed as element 29 in FIGS. 2B-2E), can be used in conjunction with a dryer/dehumidifier 9 as shown, for example, in FIG. 8A.

Where the equilibrators described herein are to be used in conjunction with a liquid reservoir 60 and carrier liquid is recirculated between the reservoir and the chamber, the temperature of the carrier liquid (e.g., water) may be controlled through the use of a reservoir temperature control element (e.g., element 61 as shown in FIGS. 8C-8D).

Apparatus comprising a gas-liquid equilibrator as described herein may comprise any combination of temperature control mechanisms. For example, an apparatus may comprise an external heating and/or cooling unit 33 or 40 for controlling the temperature of equilibration member (see, e.g., FIGS. 2D and 2E) and a liquid inlet temperature control element 27. Similarly, an apparatus comprising a liquid reservoir 60 may comprise a reservoir temperature control element 61 and a liquid inlet temperature control element 27.

### 5. Materials for Chamber and Equilibration Member

The equilibrator apparatus may be constructed of any suitable materials. Generally, the materials used for construction, particularly of the equilibration member, are not porous and do not absorb water, as trapped water could interfere with gas exchange and/or increase the time required for the gas stream passing through the equilibrator to reflect the concentration of gases in the liquid being sampled (increase the response time of the equilibrator).

Generally, the equilibrator components (e.g., the chamber, equilibration member, liquid inlet, liquid outlet, gas inlet, gas outlet, etc.) are comprised of plastics (e.g., thermoset or thermoformed polymers) and/or metals that are selected independently for each component of the equilibrator. Such plastics include, but are not limited to, acrylonitrile butadiene styrene (ABS), acrylic (e.g., polymethyl-methylacrylate), epoxy, polyamide (e.g., nylons), polycarbonate, polyester, polyether ether ketone (PEEK), polyetherketoneketone (PEKK), polyethylene (e.g., low density or high density polyethylene), polyethylene terephthalate, polypropylene, polystyrene, polysulfone, polyphenylsulfone, polyamide-imide (PAI), high impact polystyrene (HIPS), polytetrafluoroethylene (e.g., Teflon), polyvinyl chloride (PVC), polyurethane, urea formaldehyde, vinyl and combinations thereof. Where a greater degree of chemical resistance is desired, polymers such as polyetherether ketone (PEEK) or fluoropolymers (e.g., polyvinylidene fluoride (PVDF), polytetrafluoroethylene (PTFE), or fluorinated ethylene propylene (FEP)) may be employed. Any of those polymers can be reinforced (e.g., with glass fibers). Metals that may be employed include, but are not limited to, aluminum, iron, steel, stainless steel, titanium, zinc, copper and its alloys (e.g., brasses and bronzes), and combinations thereof. Metal components may be coated with a polymer coating (e.g., a polymeric powder coating), an enamel coating, a sacrificial metal coating (e.g., zinc galvanizing), antifouling coatings (e.g., coatings containing copper such as those used in marine environments), or a barrier metal coating (e.g., chrome, platinum, rhodium) to avoid corrosion. Where stainless steels are employed, they may be, for example, austenitic stainless steel (e.g., 200 or 300 series alloys), duplex grade stainless steel (e.g., high chromium low nickel such as 18% to 28% Cr and 1.5% to 8% Ni), precipitation-hardening grade stainless alloys, ferritic stainless steel, or martensitic stainless steel. In addition to the metal and polymeric materials recited above, ceramic, graphite, and glass, including borosilicate glass, may be used to prepare one or more components of the equilibrator.

Because many polymers have relatively low thermal conductivity (e.g., less than 1 W⁻¹K⁻¹), where the temperature of the equilibration reaction is controlled, one or more components of the equilibrator may be prepared from materials that are relatively thermally conductive (e.g., metals with a conductivity greater than about 10 W⁻¹K⁻¹). The metals may be selected to have thermal conductivities from 10 to 50 W⁻¹K⁻¹ or 50 to 100 W⁻¹K⁻¹. The metals may be selected to have thermal conductivities from 100 to 200 W⁻¹K⁻¹ or greater than 200 W⁻¹K⁻¹. The metals may include those with corrosion resistance, including nickel or nickel alloys, chromium or its alloys, copper or its alloys (e.g., brasses or bronzes), aluminum or its alloys, titanium, stainless steels, etc. In those conditions where an equilibrator or its components may be exposed to chemically harsh (e.g., corrosive) conditions, noble metal coatings (e.g., platinum or rhodium) may also be coated onto more highly conductive metals such as copper. For example, where high thermal conductivity of components is needed in chemically harsh environments, copper or aluminum components provided with a chemically resistant polymeric or noble metal coating can be employed.

In an embodiment the chamber is formed from polypropylene and/or polyethylene and the equilibration member is formed from polypropylene and/or polyethylene or a metal such as steel that is coated to avoid corrosion. In such an embodiment, magnetic materials may be incorporated into the equilibration member to make it susceptible to magnetic localization inside of the chamber as discussed above.

Seals, which may be used, for example, in conjunction with removably-resealable portions of the chamber wall, and with gas and liquid inlets and/or outlets, can be formed from a variety of suitable materials. Materials suitable for forming seals include, but are not limited to, natural or synthetic rubbers (e.g., silicone rubber).

Long periods of exposure to daylight can negatively impact the equilibrator. Where plastics and/or rubbers or other materials that are susceptible to photo degradation/damage are used, they may include light stabilizers including, but not limited to, antioxidants, hindered amine light stabilizers, UV absorbers and the like. In addition, exposure to light permits the growth of algae and other organisms, particularly when the liquid being tested is an aqueous liquid (e.g., fresh water, sea water and the like). Accordingly, plastics that are colored or contain fillers that substantially block or reflect light capable of supporting photosynthesis (e.g., from about 350 nm to about 750 nm) reduce the possible fouling of the equipment while extending the period between required service to keep the equilibrator clean and functioning properly. Coatings on the exterior of the chamber that reflect or absorb light can be used in place of colored plastics. Additionally, opaque fabric covers or shrouds can be used to protect the equilibrator from harmful or photosynthesis promoting solar radiation.

Where water or other aqueous liquids are subject to measurement, the chamber may be made of materials that are hydrophobic and/or omniphobic, or coated with hydrophobic and/or omniphobic coatings on all or part of the chamber's inner surface (all or part of the outer surface of the chamber may also be coated). Where non-aqueous liquids, or aqueous liquids having substantial amounts of other materials such as alcohols present, are subjected to measurement, omniphobic materials and/or omniphobic coatings may be utilized on all or part of the interior surface of the chamber (all or part of the outer surface of the chamber may also be coated). By controlling the hydrophobicity or omniphobicity of the chamber's inner surface (or the slide angle with, for example, aqueous test liquids), the response time of the equilibrator may be improved as droplets of liquid will not stick to the chamber walls. Another advantage of using a chamber with a hydrophobic or super hydrophobic inner surface is that such surfaces are considered “self-cleaning” as they resist the adherence of dirt and other materials/organisms that can foul the surface. Accordingly, the use of hydrophobic, super hydrophobic, or omniphobic surfaces extends the period of equilibrator operation between maintenance service required to keep it functioning properly (indicated by maintaining the e-folding time to within 5%, 10%, 15% or 20% of the initial e-folding time established with an unfouled (clean) equilibrator operated under the same conditions (e.g., the equilibrator's initial e-folding time value or the e-folding time value after cleaning). Self-cleaning effects are most pronounced when the surfaces are omniphobic.

In contrast to the chamber's inside walls (which may be hydrophobic or omniphobic, or substantially hydrophobic repelling water and/or substantially omniphobic repelling water and other materials) where aqueous (or polar) liquids are being assessed, the equilibration member can be made hydrophilic, thereby encouraging the film of aqueous (or polar) liquid to spread over the equilibration member's surface and increasing the surface area of the film and the exchange of gases.

In one embodiment, at least the interior surface of the chamber wall is made hydrophobic (or is made to have a low slide angle with water) and the equilibration member is made to have a hydrophilic surface. In another embodiment, at least the interior surface of the chamber wall is made super hydrophobic (or is made to have a slide angle with water less than 50) and the equilibration member is made to have a hydrophilic surface. In another embodiment, at least the interior surface of the chamber wall is made omniphobic and the equilibration member is made to have a hydrophilic surface.

In an embodiment, all or part of the interior surface of the chamber (e.g., the interior of the chamber wall that can contact an aqueous test liquid) is hydrophobic and has a contact angle with water greater than about 900 (e.g., greater than about 100°, 110°, 120°, 130°, 140°, 1500 or 160°) at 22° C.

In an embodiment, an interior surface of the chamber (e.g., an interior chamber wall that can contact a test liquid) has a slide angle with water of less than about 30° (e.g., less than about 20°, 10°, or 5°) from the horizontal (level) at 22° C. For the purpose of this disclosure the slide angle for a material is the angle at which half of a set of ten water droplets, 25 microliters in volume, slide off or to the edge of a planar piece of the material as its incline is gradually increased from the horizontal (0°). For the purposes of this disclosure, a low slide angle is less than 10°.

Where the interior of the chamber is not already hydrophobic (e.g., constructed of a material with a suitable hydrophobicity), the interior surface of the chamber may be made hydrophobic or super hydrophobic by chemical treatment or by coating it with a hydrophobic coating. In one embodiment, all or part of the surface of the chamber (e.g., all or part of the inner surface of the chamber wall) is modified by treatment with hydrophobic silanizing agents (e.g., alkyl and fluoroalkyl silanizing agents). Hydrophobic silanizing agents include, but are not limited to: (tridecafluoro-1,1,2,2-tetrahydrooctyl) trichlorosilane; (tridecafluoro-1,1,2,2-tetrahydrooctyl) triethoxysilane; (tridecafluoro-1,1,2,2-tetrahydrooctyl) trimethoxysilane; (heptadecafluoro-1,1,2,2-tetrahydrodecyl)dimethyl(dimethylamino)silane; n-octadecyltrimethoxysilane; n-octyltriethoxysilane; and nonafluorohexyldimethyl(dimethylamino)silane. In other embodiments, all or part of the interior surface of the chamber may be treated with a hydrophobic coating to render the treated surfaces hydrophobic or super hydrophobic. Hydrophobic coatings include those with polyurethane, acrylic, and fluorovinyl polymer systems (see, e.g., U.S. Pat. Nos. 5,962,620 and 9,067,821). Where it is desirable to have omniphobic behavior, the silanizing agents and/or coatings (e.g., the polymers of the coatings) should comprise fluoroalkyl groups.

In an embodiment, where the liquid subject to testing is an aqueous liquid, the surface of the equilibration member may be made hydrophilic. In such an embodiment, the contact angle of the equilibration member with water may be less than about 600 (e.g., less than about 50°, 40°, 30°, 200 or 10°) at 22° C. As discussed above, contact angles are measured using a goniometer.

If the equilibration member is not already hydrophilic (e.g., constructed of a material with a suitable hydrophilicity), the surface of the equilibration member may be made hydrophilic by chemical treatment or by coating it with a hydrophilic coating. In one embodiment, the hydrophilicity of the equilibration member is modified by treatment with hydrophilic silanizing agents. In another embodiment, the surface of the equilibration member (e.g., rubber or plastic) may be treated with a plasma (e.g., oxygen plasma) to provide hydroxyl, carboxyl and carbonyl groups. In other embodiments, oxygen plasma treated surfaces are subsequently treated with a nitrogen plasma to affix nitrogen containing groups to the surface and render it more hydrophilic. In other embodiments, all or part of the interior surface of the chamber may be treated with a hydrophilic coating (e.g., hydrophilic polyurethane, acrylic, or hydrogel compositions, etc.) to render the treated surfaces hydrophilic (see, e.g., U.S. Pat. No. 5,962,620 or U.S. Pat. No. 6,017,577 describing hydrogels).

For the purpose of this disclosure, materials or surfaces are considered to be hydrophobic when the static contact angle of the surface with water at 22° C. is 900 or greater. Surfaces are considered to be super hydrophobic when the static contact angle with water at 22° C. is greater than 150°. Surfaces are considered omniphobic when they have a static contact angle with both water and hexadecane greater than 90° at 22° C. For the purpose of this disclosure, materials or surfaces are considered to be hydrophilic when the static contact angle of the surface with water at 22° C. is less than 90°. Contact angles (static contact angles) are measured using a goniometer (e.g., Attension Model Theta Flex goniometer, available from Biolin Scientific, formerly KSV Instruments, Stockholm, Sweden) according to the manufacturer's instructions.

### 6. Operation of the Equilibrator

In general terms, the equilibrator operates by having liquid introduced in the upper portion of the equilibrator chamber such that it contacts the equilibration member forming a film that is drawn downwards over the equilibration member (a falling film) to the lower portion of the chamber where it is directed to a liquid outlet and leaves the equilibrator. At the same time liquid is introduced into the upper portion of the equilibrator, a carrier gas is introduced into the lower portion of the chamber. Once introduced into the lower portion of the chamber, the incoming gas is displaced upward by the stream of incoming carrier gas. As carrier gas moves upward it contacts the falling film of liquid and the gases (e.g., carbon dioxide) in the liquid exchange into the carrier gas progressing toward equilibrium concentration as the liquid and carrier gas move in a counter current manner. The carrier gas, which is near or has reached equilibrium with the gases in the incoming liquid, ultimately reaches the upper portion of the chamber where it exits the chamber via the gas outlet(s). After exiting the chamber via the gas outlet(s), all or part of the carrier gas is directed to the sensor of an analytical instrument (gas analyzer) that can measure the amount of the gas of interest in the carrier gas.

Where the liquid is water or an aqueous solution, systems that incorporate the equilibrator with an analytical instrument may also have a dryer/dehumidifier interposed between the gas outlet(s) of the equilibrator and the sensor to remove from the carrier gas any liquid vapor (e.g., water vapor) and/or any liquid that condenses (e.g., water) in the gas outlet line (sample gas line 8 connected to gas outlet 4) before the carrier gas reaches the sensor 16 of the analytical instrument 17. Equilibrated sample gas may be pulled (e.g., using reduced pressure or a slight vacuum) through the gas outlet line, through the dryer/dehumidifier, and into/through the gas sensor by the intake side of a gas/air pump (e.g., a vane, fan, diaphragm, etc.) 23 that is located downstream from the sensor. Carrier gas is directed from the pump outlet under positive pressure into the gas inlet line leading to the equilibrator. The dryer/dehumidifier will generally be placed “upstream” of the sensor of the gas analyzer when the system is operating in the forward direction (forward flow causes carrier gas to move in the direction from the equilibrator's gas outlet toward the analytical instrument's sensor, reverse flow takes gas in the opposite direction toward the equilibrator's gas outlet). The dryer/dehumidifier 9 may comprise one or more of a water trap 10, a filter 11 (e.g., a membrane filter made of paper, nylon, polyvinylidene difluoride (PVDF) and the like), and/or a drying tube assembly 12. The drying tube assembly may comprise a dehumidifying Nafion® polymer tube 13 that is supplied with a flow of drying gas (e.g., air) through a drying gas inlet 14 and drying gas outlet 15 that has a lower amount of water vapor such that it can dehumidify/dry the carrier gas stream coming from the equilibrator.

In an embodiment, carrier gas (e.g., air or an inert gas) exiting the chamber via the gas outlet(s), along with the gas of interest and liquid vapor (e.g., water vapor), is recirculated back to the gas inlet(s) after passing through one or more sensors of the analytical instrument 16 and the dryer/dehumidifier 9 if present. The gas may thus be kept in a closed carrier gas loop except, for example, during periods when all or part of it is replaced or displaced by fresh carrier gas or when a gas standard is used to calibrate the analytical instrument. Analytical instrument 17 may contain a single type of sensor (e.g., CO₂) or multiple sensors arranged in parallel and/or in series that detect different species within the carrier gas stream (e.g., CO₂, methane, radon, etc.). Accordingly, different species can be detected using the same equilibrated sample gas, either by placing sensors in series within a single gas train or in parallel where the gas train has been split after leaving the equilibrator and rejoined prior to entering the equilibrator gas inlet. In an embodiment, the analytical instrument contains at least a first sensor that is arranged in parallel with a second sensor, and a third sensor in series with the first sensor.

Where the liquid subject to analysis forms as condensate (e.g., aqueous solutions or water), it may be desirable to periodically reverse the flow of gas in the line (tubing) attached to the gas outlet so that liquid that has been swept into and/or condensed in the lines is carried back into the chamber and to remove liquid from the dryer/dehumidifier 9. In some embodiments, the gas flow may be reversed through a segment of the line 8 proximate to the gas outlet of the chamber passing through the dryer/dehumidifier 9 (if present) and exhausted at port 19 or, after passing through the chamber at port 22 (from sampling port 20 which is exhausted at port 19 or 22). In other embodiments, the flow may be reversed through both the dryer/dehumidifier 9 and the sensor 16 (e.g., gas flow from sample port 21 which is exhausted at port 19 or 22). Valves 19a, 20a, 21a, and 22a are capable of connecting and closing off any combination of lines connected to them, but when measurements of a gas of interest in a liquid sample are being made they close off only the line to ports 19, 20, 21, and 22. Where gas circulation is reversed through the chamber it can be advantageous to stop the flow of liquid into the chamber during the period of reverse flow using a valve 18 upstream of liquid inlet 1.

Where gas flow is reversed for the purpose of clearing the gas lines of condensed liquids and/or drying the gas lines, previously unused carrier gas or gas used for standardization/calibration of the equipment (e.g., air or a gas with a known CO₂ or other gas species concentration) may be directed into the system (e.g., via port 20 or 21) at one end of the section of the equipment to be dried and/or calibrated, and allowed to exit at a point downstream of the portion subject to drying and/or calibration.

In addition to the foregoing, to avoid the buildup of analyte(s) in the gas phase it may be desirable to replace all or part of the carrier gas in an apparatus comprising a gas-liquid falling film equilibrator described herein and a closed or substantially closed carrier gas loop. This is particularly true where the apparatus comprising a gas-liquid falling film equilibration system is configured for discrete sample analysis with closed or substantially closed carrier gas and carrier liquid loops. The carrier gas can be replaced in whole or in part using ports in the carrier gas loop (see, e.g., ports 19-22 in FIGS. 8A-8D).

In view of the foregoing, in one embodiment, an apparatus comprising an equilibrator as described herein may be operated to determine the amount of one or more gases and/or one or more volatile substances of interest present in a liquid (e.g., a test liquid or a standard having a known or added amount of an analyte gas or volatile substance of interest) employing a method comprising the steps:
i) providing an apparatus of any one of the aspects enumerated below (e.g., aspects 1-32 or aspects) in the section titled “Certain Aspects”;
ii) introducing the liquid at an initial temperature (T1) into the chamber of the apparatus by way of the liquid inlet beginning at an initial time (t1) of a first time period such that it passes over the equilibration member thereby forming a falling film over all or part of the equilibration member's surface, and exits the apparatus by way of the liquid outlet;
iii) continuing the introducing of the liquid during the first period of time while operating the temperature control mechanism to stabilize (maintain) the temperature of the liquid in a range that (a) encompasses the initial temperature (T1), (b) is in a range above the initial temperature, or (c) is in a range that is below the initial temperature;
iv) during the first period of time, directing a carrier gas into the chamber of the apparatus by way of the gas inlet such that it flows over the equilibration member in a direction that is counter current or substantially counter current to the flow of the liquid, and exits the chamber of the apparatus by way of the gas outlet (“the gas that exits the chamber”); and
v) during all or part of the first period of time, directing all or part of the gas that exits the chamber to a sensor that produces output related to the amount of the one or more gases and/or one or more volatile substances (e.g., the sensor of an analytical instrument capable of detecting and/or measuring the amount of the one or more gases and/or one or more volatile substances).
   The method may further comprise as step vi—determining the amount of the one or more gases and/or one or more volatile substances present in the liquid during the first period of time based on the output of the sensor.

The initial temperature (T1) is the ambient temperature of the liquid to be analyzed (test liquid) in the environment from which it is being taken at the starting time (t1) of a period of time when the sample is introduced into the chamber for a determination of the amount of a gas or volatile substance (step ii of the method outlined above). In the case of a series of measurements, T1 is the ambient temperature of the liquid to be analyzed at the starting time (t1) of the first period of time for which a determination is to be conducted unless stated otherwise. The ambient temperature of the liquid being analyzed in any period of time subsequent to the first period of time (e.g., a second time period, a third time period etc.), although relevant to the process, does not change the value of T1. For example, T1 may be the temperature of a lake, ocean, river, reservoir, pool, retention pond, etc., at the start of the first period of time when a measurement or series of measurements is made. Where the temperature of the liquid entering the equilibrator chamber at liquid inlet 1 has not been substantially altered from its ambient temperature, for example by operation of a liquid inlet temperature control 27, the temperature of liquid at the liquid inlet to the chamber at t1 may serve as a surrogate for T1.

Steps ii to v represent the portion of the process where sampling of a liquid being analyzed occurs. The determining step, step vi, may be conducted concurrently with the sampling as in the case where inline sensors with any associated analytical instrumentation (including required computers) are used to examine the stream of gas exiting the chamber. In that case the output from the sensors can be used to produce real time readings of at least one of the one or more gases and/or one or more volatile substances in the liquid.

Determinations of at least one of the one or more gases and/or one or more volatile substances in the liquid can also be conducted, in whole or in part, separately from the sampling steps. In one case, determinations of the amount of one or more gases and/or one or more volatile substances present in the liquid based on the output of a sensor (sensor data) may be made at a later time and/or location by transmitting (e.g., via an electronic or optical link to the internet, cellular telephone network or satellite) or by storing the output of the sensor on electronic storage media. The output of the sensor can be assessed at a later time and/or location to produce determinations of at least one of the one or more gases and/or one or more volatile substances present in the liquid.

The step of directing all or part of the gas that exits the chamber to one or more sensors (step v) encompasses obtaining discrete samples of the gas exiting the chamber (e.g., for the first and any subsequent time period desired) that can later be directed to the one or more sensors. Accordingly, any (e.g., some) of the one or more sensors and any analytical instrumentation and/or computers need not be physically connected to the equilibrator for the determining step (vi), which may be conducted on the discrete samples at a later time in a separate location. Where gas analysis is conducted on discrete samples removed from a system using a closed or substantially closed carrier gas loop, an amount of gas equal to that removed can be introduced into the carrier gas to replace the amount taken in the discrete sample.

In some instances, the periods of time over which gas exiting the chamber is directed to the sensor for determination of gas(es) or volatile substance(s) (the periods of time for determinations) may be less than about 10 seconds or less than about 1 minute. In other instances, the periods of time may be greater than about 1 minute and less than about 6 minutes, or greater than about 6 minutes and less than about 1 hour. Each period of time for a determination may be selected independently. In some instances, the period of time for any one or more determinations (each period of time) may be equal. In some instances, the period of time for any one or more (each) determination subsequent to the first determination is less than or equal to the first period of time. In some instances, the period of time for any one or more (each) determination subsequent to the first determination is greater than or equal to the first period of time.

The periods of time over which gas exiting the chamber is directed to the sensor for determinations (periods of time for determinations) of the amount of one or more gases and/or one or more volatile substances need not run in an uninterrupted succession (back-to-back). Intervals of time between the periods of time for determinations may be included in the process. The intervals between any two periods of time for determinations may be selected independently. The intervals between any two periods of time for determinations may be equal, equal within a range (e.g., +/−10% or +/−5%), or substantially equal. During the intervals between periods of time for determinations, introducing of the liquid into the chamber may be continued. Alternatively, the flow of liquid into the chamber may be stopped during intervals between determinations, for example to conserve energy in remote installations where power may be limited, provided the equilibrator inlets and outlets (e.g., liquid outlet 2) are sealed such as by a valve or liquid trap.

During any one or more periods of time for determinations of gases and/or volatile substances, one or more temperature control mechanisms may be operated to stabilize (maintain) the temperature of the liquid in a temperature range. The temperature may be maintained during all or part of the first period of time and/or during all or part of any (e.g., each) subsequent period of time for determinations. Where ranges extend above the boiling point or below the freezing point, that portion of the range is understood to be excluded (the range is truncated at the boiling and/or freezing point) as the liquid being analyzed is no longer a liquid under those circumstances.

The temperature of the liquid may be maintained in a range that encompasses the initial temperature (T1). The temperature of the liquid may be maintained in a range that is +/−20° C. or +/−10° C. of the initial temperature (T1). The temperature of the liquid may be maintained in a range that is +/−5° C. or +/−2.5° C. of the initial temperature (T1).

The temperature of the liquid may be maintained in a range above the initial temperature (T1). The temperature of the liquid may be maintained in a range that is less than 10° C. above the initial temperature (T1) or 10-20° C. above the initial temperature (T1). The temperature of the liquid may be maintained in a range that is 20-30° C. above the initial temperature or more than 30° C. above the initial temperature (T1).

The temperature of the liquid may be maintained in a range below the initial temperature (T1). The temperature of the liquid may be maintained in a range that is less than 10° C. below the initial temperature (T1) or 10-20° C. below the initial temperature (T1). The temperature of the liquid may be maintained in a range that is 20-30° C. below the initial temperature or more than 30° C. below the initial temperature (T1).

When analyzing discrete samples using an apparatus comprising an equilibrator described herein, the samples may be introduced into the chamber of the apparatus using an injector located upstream of the liquid inlet (see, e.g., FIGS. 8B-8D). The injector may be a section of tubing or a septum (e.g., prepared from silicone rubber or silicone rubber/PTFE composites) that can be pierced by a needle and can be self-healing (substantially or completely reseals after penetration by a non-coring needle). Alternatively, the injector may use a sample loop (see, e.g., 53 in FIG. 8E) into which a sample is loaded and then the sample loop is interposed into the tube carrying the stream of liquid directed to the liquid inlet 1 thereby causing the liquid sample to enter the chamber. Use of a sample loop permits operation of the apparatus by recirculating a set amount of carrier liquid without changing the carrier liquid volume because the sample liquid displaces from the sample loop an equal volume of carrier liquid when the sample is loaded. An example of a suitable injector 50 that uses a sample loop 53, is the six port injector depicted in FIG. 8E. The incoming liquid stream enters the injector through port 54 and exits the injector through port 55. When the injector is in the loading position (FIG. 8E at A) it permits sample to be introduced to the sample (injector) loop 53 through injector port 51 which is in fluid communication with sample loop 53 and overfill port 52. Rotating hub 56 relative to the ports directs the incoming liquid stream entering the injector by port 54 to the sample loop which exits the injector through port 55 and is then directed to the equilibration chamber.

An apparatus for analyzing discrete samples of liquid (test liquid) may be operated to determine the amount of one or more gases and/or one or more volatile substances present in a sample of test liquid employing a method comprising the steps:
i) providing an apparatus comprising an equilibrator as described hereinabove (or an apparatus of any one of aspects 34-59 set forth in the disclosure that follows), wherein (a) a volume of carrier liquid at a temperature T is recirculated through a closed or substantially closed carrier liquid loop comprising the liquid inlet 1, chamber c, the liquid outlet 2, sample injector 50, and liquid reservoir 60, while optionally operating a temperature control mechanism to stabilize (maintain) the temperature of the liquid in a temperature range, and (b) a volume of carrier gas is recirculated through a closed or substantially closed carrier gas loop comprising the gas inlet 3, chamber c, gas outlet 4, and headspace 63 of the liquid reservoir in a direction that is counter current or substantially counter current to the flow of the carrier liquid, and exits the chamber of the apparatus by way of the gas outlet 4;
ii) introducing a sample of the test liquid (a sample of discrete known volume) into the carrier liquid by way of a sample injector 50, the sample of test liquid and carrier liquid entering the chamber c of the apparatus by way of the liquid inlet 1 such that it passes over the equilibration member, thereby forming a falling film over all or part of the equilibration member's surface, and exits the apparatus by way of the liquid outlet 2; and
iii) directing all or part of the gas that exits the chamber through gas outlet 4 to one or more sensors that produces an output corresponding to the amount of at least one of the one or more gases and/or one or more volatile substances present in the liquid (e.g., the sensor(s) of an analytical instrument capable of detecting and/or measuring the amount of the one or more gases and/or one or more volatile substances).

The method may further comprise as step iv—based on the output of the one or more sensors, determining the amount of the one or more gases and/or one or more volatile substances present in the sample of test liquid. As with apparatus comprising an equilibrator described herein used for analyzing non-discrete samples, when analyzing discrete samples the determination of at least one of the one or more gases and/or one or more volatile substances in the liquid can also be conducted, in whole or in part, at a separate time and/or location from the sampling steps (including obtaining sensor readings). For example, output of a sensor (sensor data) may be analyzed to determine gas concentrations at a later time and/or location after having been transmitted (e.g., via an electronic or optical link to the internet, cellular telephone network or satellite), or by storing the output of the sensor on electronic storage media.

The temperature T may be at, above, or below the ambient temperature at which the apparatus used for discrete sample analysis is operated. The temperature of a carrier liquid may be adjusted to promote the gas or volatile component of interest exiting the liquid and entering the gas phase by operating the apparatus at a temperature (e.g., carrier liquid temperature) where the gas or volatile component is less soluble in the liquid.

In some embodiments, in addition to the equilibrator, the sensor of the analytical instrument, and an optional dryer/dehumidifier, the system may comprise an auto-controlled drying mechanism and carrier gas (e.g., atmospheric gas/air) sampling port circuit composed of a combination of solenoid and valves (e.g., one-way valves) and an electrical relay to simultaneously stop water pumping into the equilibrator. In such embodiments, the method may further comprise the steps of sampling carrier gas (e.g., air from the atmosphere) through the carrier gas sampling port 21 (by opening a valve to that port) and directing it to the sensor for measurement/calibration purposes, after which it is exhausted toward the equilibrator through the same sample gas line that connects the gas out of the equilibrator chamber to the sensor system. By doing so, the line that normally brings gas laden with liquid vapor (e.g., water vapor) from the equilibrator to the sensor system can be cleared of accumulated liquid that has, for example, condensed in the line and the sensor system. By directing carrier gas that is not saturated with liquid vapor (e.g., water vapor) from the sampling port 21 through the dryer/dehumidifier, the dryer/dehumidifier apparatus and/or any chemical drying agents it contains may be fully or partially regenerated. Alternatively, the chemical drying agent may be contained in a chamber equipped with a heating element and may be periodically regenerated by heating the drying agent if the apparatus is located where energy consumption of the drying process can be provided. In an embodiment, the chemical drying agent is replaced periodically at times determined by the climate/ambient relative humidity and temperature instead of being regenerated.

The frequency with which the flow of gas is reversed to remove all or part of the liquid that might accumulate in the lines (tubing) carrying gas from the equilibrator to the sensor system can vary depending on a variety of factors. Fluid accumulation in the line leading from the gas outlet of the chamber to the sensor is often the result of condensation of vapor from the fluid being sampled becoming part of the carrier gas stream. Accordingly, the temperature of the fluid, which will change its vapor pressure, and the temperature of the line, which is largely dictated by ambient temperature of the location where the sensor part of the system is installed, may in large part dictate the need for clearing the line of fluid. In embodiments, the direction of gas flow is reversed to clear the lines during less than 25% (e.g., less than 20, 15, 10 or 5%) of its operating time. By way of example, a system encompassing the equilibrator may have the direction of gas flow reversed for a continuous period of 15 minutes every one, two, three, four, five, or six hours. Under field conditions, 15 minutes per six hours operating time is often sufficient and provides the opportunity to measure ambient atmospheric gas concentrations (e.g., ambient pCO₂ values).

As discussed above, the equilibrator functions by permitting the exchange of the gas of interest between the film of fluid being drawn downward over the equilibration member (falling film) and the gas moving up through the equilibrator. Accordingly, efficient exchange requires the film of liquid have sufficient area. At the same time, the gas flow should be sufficient to provide a suitable response time, but not so fast as to cause turbulence in the equilibrator (e.g., turbulence that carries water droplets into the gas outlet(s)). The flow of liquid into the equilibrator required to provide a film of sufficient area depends on many factors including, but not limited to, the shape of the equilibration member, its dimensions (including surface area), the viscosity of the liquid, and the interaction between the liquid and the surface of the equilibration member (e.g., is there enough interaction energy between the surface of the equilibration member and the liquid for efficient wetting). In general, falling films are initiated with liquid of sufficient volume that is injected with some positive pressure onto the top surface of the equilibration member to completely wet the surface of the equilibration member and to maximize the integrated wetted surface area over time. Larger equilibration members will have more instantaneous wetted surface area than smaller equilibration members.

In an embodiment, the flow of liquid required to maintain a falling film over the surface of the equilibration member may vary from about 0.25 liters/minute (l/min) to about 12 l/min (e.g., 0.25-1, 0.25-2, 1-4, 2-6, 4-8, 6-12 or 8-12). Exact flow rates will be limited by the equilibrator member surface area and drain diameter, and thus may have potential for a broad working range. Lower flow rates, such as 0.25 or 1.0 l/min, are useful with smaller equilibration members (e.g., those with surface areas of less than 1000 cm²) and higher flow rates, such as 6-12 or 8-12 l/min, with larger equilibration members (e.g., those with surface areas of 1000 cm² or greater).

Gas flow rates through the chamber during operation necessary to obtain measurements will vary depending on a variety of factors including, but not limited to, the interior volume of the chamber, the shape of the chamber, and the desired response time of the apparatus to changes in the content of a gas of interest in the liquid being sampled. In an embodiment, the gas flow may vary from about 0.1 liters/minute (l/min) to about 3 l/min (e.g., 0.1-1, 1-2, or 2-3 l/min). Flow rates (e.g., in cm³/min.) may be adjusted based on the chamber's headspace (interior volume not occupied by the equilibration member or support structures such as annular rings and/or pedestals), with lower flow rates of about 0.08 to about 2.5 cm³ of carrier gas per cm³ of headspace per min. (e.g., from about 0.08 to about 0.2, from about 0.2 to about 0.5, from about 0.5 to about 1.0, from about 1.0 to about 2.0, or from about 2.0 to 2.5 cm³ of carrier gas/cm³ of headspace per minute).

For operation the equilibrator apparatus described herein can be mounted to a stationary mount. Alternatively, because the equilibrator can operate when tipped at moderate angles, it can be mounted on a mobile platform such as a boat, buoy, raft or similar platform, permitting a range of installation options for measuring gases of interest. The equilibrator is not disturbed by bubbles or particulates small enough to pass through the lines/nozzles used to deliver liquids to the chamber and/or the fluid outlet and lines that carry liquid away from the equilibrator's chamber.

Because various gas species are produced through both natural and engineered processes, measurements of gas concentration are important for understanding many aspects of water quality. There are many applications for equilibration and measurement that address both environmental and human health issues. Virtually any gas species that can be absorbed in water (natural surface waters, pore water, groundwater/aquifer, or water within engineered water systems such as wells, waste water treatment facilities, drinking water treatment plants, swimming pools, and algal photobioreactor systems used for carbon capture and sequestration, etc.) can be equilibrated or substantially equilibrated with carrier gas or air in the chamber's headspace of the falling film equilibrators described herein regardless of the relative solubility of the gas. Gas species can then be measured by the use of the appropriate analytical instrument and sensor (e.g., NDIR, photo-acoustic detectors, gas chromatographs, radiation such as alpha particles) in either real time or as discrete samples.

Among the gases that could be measured using the falling film equilibrators described herein are ammonia, CO₂, CO, sulfur oxides (e.g., sulfur dioxide), nitrogen oxides (e.g., NO or NO₂), methane, ethane, hydrocarbons, halogenated hydrocarbons, chlorofluorocarbons (CFCs), hydrofluorocarbons (HFCs), perfluorocarbons (PFCs), esters, sulfur hexafluoride (SF₆), chlorine, bromine, radon, hydrogen sulfide (H₂S), HF, HCl, HBr, and Hl. Measurements can be made, for example, of one, two, three or more of such gases. By way of example, measurements of CO₂ can be made by infrared detection and measurements of radon by using a detector for alpha-radiation. Some gases/volatile materials particularly relevant to human health and/or of environmental concern that can be measured in, for example, aqueous samples using the falling film equilibrator described herein include carbon dioxide, methane, radon, hydrogen sulfide, halogenated alkanes (e.g., trihalomethanes), sulfur hexafluoride, nitrous oxide, and sulfur dioxide.

Carbon dioxide (CO₂) can be measured to determine its concentration as related to carbonate chemistry (the chemistry of ocean acidification comprised of total dissolved inorganic carbon, carbonate, bicarbonate, pH, total alkalinity, etc.), CO₂ sources/sinks (e.g., estuaries, rivers, streams), pCO₂/pH control (e.g., monitoring and control of pH in swimming pools), ecosystem metabolism (e.g., photosynthesis/respiration patterns), and carbon capture/sequestration in industrial settings, and in understanding greenhouse gas effects.

Methane (CH₄) is an important gas to monitor as it is both a greenhouse gas with 25× the forcing potential of CO₂ and explosive if it builds up to significant levels. Methane can occur in drinking water, wastewater, groundwater/aquifers, and pore water in natural aquatic systems (e.g., lakes, rivers, streams, wetlands), in engineered environments such as industrial ponds, and in water released from industrial processes and engineered environments. Sources of methane include industrial (e.g., petroleum) processing, natural gas release, and agricultural sources (livestock and manure).

Radon (Rn) is a human health hazard linked to the development of lung cancer that is produced naturally via the radioactive decay of uranium in bedrock and occurs in well water, aquifers, rivers, the sump of numerous homes, etc.

Hydrogen sulfide (H₂S) is a poisonous, corrosive, flammable gas produced by anaerobic microbial decomposition of organic materials in wetlands and sewers, and also occurs in natural gas and volcanic gases.

Halogenated alkanes, including total trihalomethanes (e.g., chloroform (CHCl₃), bromoform (CHBr₃), dibromochloromethane (CHBr₂Cl), and bromodichloromethane (CHBrCl₂), are a human health hazard due to their toxicity. Trihalomethanes are common water disinfection byproducts resulting from water chlorination. While the concentration of halogenated alkanes is regulated in drinking water, they occur commonly in swimming pools.

Sulfur hexafluoride (SF₆), which is used as a tracer gas and an electrical insulator, represents a substantial environmental hazard. Sulfur hexafluoride is one of, if not the, most potent greenhouse gases, as evaluated by PICCC (Primary Industries Climate Challenges Centre), having 22,000× the forcing potential of CO₂.

Nitrous oxide (N₂O) is an environmentally hazardous material that can contribute to greenhouse warming (298× the forcing potential of CO₂). Nitrous oxide is produced naturally by microbial processes in soils, manure, and the ocean. The gas also results from anthropogenic sources such as fertilized soils. It is used extensively as an aerosol propellant, in medical and dental procedures as an anesthetic, and as a supplementary oxidizer for internal combustion engines and in rocket fuel.

Sulfur dioxide (SO₂) is a major air pollutant that impacts human health. It is a precursor to inorganic acids and a component of acid rain. Sulfur dioxide has its environmental origins in volcanic sources and in the industrial combustion of sulfur containing materials.

A large variety of liquids can be assessed for the levels of dissolved gases and/or volatile substances including salt water, sea water, brackish water, tidal water, marsh water, river water, lake water, stream water, spring water, ground water, aquifer water, pore water, geyser water, volcanic water, well water, swimming pool water, aquarium water, sewage (e.g., sewer water), industrial waste streams, industrial waste water, irrigation water, run-off from agricultural sites, run-off from mines, run-off from industrial sites, drinking water, treatment plant water, and treated sewer water.

The design of the equilibrator permits monitoring of one or more gas species in a continuous or semi-continuous fashion (continuous, except during intervals where the equilibrator is operated with gas flow in the reverse direction to clear liquid), as opposed to taking discrete samples which are subject to analysis. It is also possible to incorporate additional sensors into the equilibrator or the adjacent analytical equipment to measure the characteristics of the fluid being measured such as temperature and pH, which can be measured in the body of liquid subject to testing, in the chamber, or in the lines (tubing) connected to the equilibrator.

Operation of the equilibrator and the system it is connected to for the analysis of gases of interest in liquid samples requires controlling the flow of both liquids and gases. Liquids may be directed to flow by the use of any suitable pump including, but not limited to, vane, impeller, piston, centrifuge and diaphragm pumps, any or all of which may be reversible. Similarly, the flow of gases may be directed by the use of pumps including, but not limited to, vane, impeller, piston, centrifuge, bellows, and diaphragm pumps, any or all of which may be reversible. Gases may also be directed to flow by use of a pump or a source of previously compressed gas (e.g., a pressurized tank), or by the application of reduced pressure (vacuum or partial vacuum). In operation, the movement of gases may be directed in a system incorporating an equilibrator using any combination of pumps, vacuum and compressed gas sources. The gas pump may be part of an analytical instrument or may be separate (independent) of any analytical instrument. Pumps for liquid (e.g., carrier liquid) and gas (e.g., carrier gas) are not shown, but may be positioned in suitable locations to effect the operation of the system. Where the carrier gas loop passes through the sensors of analytical instruments arranged in series or parallel, one potential location for a carrier gas pump to be located is between the instruments and the chamber. Where the carrier gas loop is divided and sent to sensors placed in parallel, a single pump can be located downstream of the sensors at a point after the carrier gas streams are rejoined in a single line. Pumps should be selected so they do not introduce materials that interfere with analyte concentration (e.g., by contamination). In addition, pumps should be substantially or completely leak free so as to avoid the loss or contamination of sample, carrier liquid, or carrier gas that would affect measurement of an analyte of interest.

### Certain Aspects

1. An apparatus comprising:
a chamber comprising an outer wall that is disposed substantially symmetrically about a central axis, the outer wall defining an interior surface of the chamber, an exterior surface of the chamber, and space within the chamber;
an equilibration member within the chamber having an equilibration member outer surface, an axis of rotation, and a bisecting plane perpendicular to the axis of rotation positioned at the midpoint of the equilibration member's axis of rotation;
the equilibration member being positioned within the chamber such that its axis of rotation and the central axis of the chamber coincide or substantially coincide;
the chamber, the exterior surface of the chamber, the interior surface of the chamber, the equilibration member and its outer surface, and the space within the chamber being divided into an upper portion above the bisecting plane and a lower portion below the bisecting plane;
the space within the upper portion of the chamber being in liquid (fluid) and gas communication with the space within the lower portion of the chamber via one or more gaps between the equilibration member and the interior surface of the chamber;
at least one (i) temperature control mechanism and/or (ii) sample injector;
a liquid inlet located in the upper portion of the chamber positioned such that a liquid introduced into the chamber through the liquid inlet contacts the upper portion of the equilibration member outer surface;
a liquid outlet located in the lower portion of the chamber positioned such that some or all of the liquid introduced into the chamber that collects in the lower portion of the chamber (e.g., by gravity) may exit the chamber as outflow;
a gas inlet located in the wall of the lower portion of the chamber; and
a gas outlet located in the wall (e.g., in the removably-resealable portion of the chamber) of the upper portion of the chamber;
   wherein at least a section of the upper portion of the chamber wall is removably-resealable to the remainder of the upper portion of the chamber wall (e.g., the upper surface), and/or the outer wall.

2. The apparatus of aspect 1 wherein the equilibration member is selected from the group consisting of: a spheroid; an ellipsoid; an ovoid; a hemisphere; a hemiellipsoid; a hemiovoid; a domed frustum; a series (two, three, four, or more) of spheres or disks aligned along the central axis (see, e.g., FIGS. 5-6); a column; a column having one, two, three, four, or more spiral grooves; a column having sinusoidal oscillating sides; a cone having one, two, three, four, or more spiral grooves; and a cone having sinusoidal oscillating sides.

3. The apparatus of any preceding aspect wherein the interior and/or exterior surface of the chamber is substantially in the form of a vertical right cylinder, a sphere, an ellipsoid, or an ovoid.

4. The apparatus of any preceding aspect, wherein the chamber is substantially in the form of a vertical right cylinder (VRC) wherein the wall forms an upper and a lower surface positioned substantially perpendicular to the central axis of the chamber.

5. The apparatus of any preceding aspect, wherein the section of the upper portion of the wall that is removably-resealable forms a lid on the remainder of the upper portion of the chamber, wherein, when the chamber is a VRC with an upper surface positioned substantially perpendicular to the central axis of the chamber, the lid comprises all or part of the planar upper surface.

6. The apparatus of aspect 5, wherein the liquid inlet and/or gas outlet are positioned in the lid.

7. The apparatus of any preceding aspect, wherein the liquid inlet is positioned either at, or proximate to, the central axis.

8. The apparatus of any preceding aspect, wherein (i) the liquid outlet and/or gas inlet are positioned in the lower portion of the outer wall of the chamber, (ii) the chamber is a VRC with a lower surface positioned substantially perpendicular to the central axis of the chamber and the liquid outlet and/or the gas inlet are positioned in the lower surface of the chamber, or (iii) the chamber comprises a substantially conical or convex lower surface and the liquid outlet is positioned in the lower surface (e.g., substantially in line with the central axis of the chamber, see, e.g., FIG. 4 and FIG. 6 a-d and o).

9. The apparatus of any preceding aspect wherein the apparatus comprises a sample injector.

10. The apparatus of aspect 9, wherein the sample injector comprises:
(i) a section of tubing that is self-healing;
(ii) a self-healing septa; or
(iii) a sample loop (injector sample loop) 53.

11. The apparatus of any preceding aspect, wherein the gas outlet is positioned in the removably-resealable portion of the chamber wall (e.g., in the flat upper surface of a VRC lid).

12. The apparatus of any one of aspects 1-10, wherein, when the chamber is a VRC, the gas outlet is not located in the removably-resealable portion of the chamber wall.

13. The apparatus of any preceding aspect, wherein the liquid inlet comprises a liquid inlet nozzle (e.g., a piece of tubing) that extends into the chamber.

14. The apparatus of any preceding aspect, wherein the liquid inlet nozzle extends into the chamber to a point (level) above the equilibration member outer surface.

15. The apparatus of any preceding aspect, wherein the equilibration member or the equilibration member outer surface is not porous and/or does not absorb water.

16. The apparatus of any preceding aspect, wherein the surface of the equilibration member is hydrophilic.

17. The apparatus of any preceding aspect, wherein the interior surface of the chamber has a contact angle with water greater than about 70°, 80°, 90°, 100°, 110°, 120°, 130°, or 140° at 22° C. or is hydrophobic with a contact angle greater than about 90° at 22° C.

18. The apparatus of any preceding aspect, wherein the interior surface of the chamber has a slide angle with water less than about 30°, 20°, 10°, or 5° at 22° C.

19. The apparatus of any preceding aspect, wherein the gas inlet comprises an opening that directs the incoming gas in the direction of the central axis or into a plane that is perpendicular to the central axis of the chamber.

20. The apparatus of any of aspects 1-19, wherein the gas inlet comprises an opening that directs the incoming gas substantially in a plane that is perpendicular to the central axis (e.g., forcing the gas to circulate in a clockwise or counterclockwise fashion within the chamber).

21. The apparatus of any preceding aspect, wherein:
i) the equilibration member is free to float on liquid that accumulates in the lower portion of the chamber (the accumulated liquid acts as a liquid bearing and the equilibration member may freely rotate under the force of the liquid entering the chamber such as via the inlet nozzle(s) described in aspects 13 and 14);
ii) the apparatus further comprises an annular element within the chamber in contact with the lower portion of the chamber (e.g., the substantially planar lower surface of a VRC) and the equilibration member; or
iii) the equilibration member is supported by one or more projections extending inward from the equilibration chamber wall and/or outward from the equilibration member.

22. The apparatus of any preceding aspect, wherein the equilibration member comprises a magnet or a magnetically susceptible material, and wherein the apparatus further comprises a magnet or magnetically susceptible material positioned on or in the chamber wall so as to magnetically engage the equilibration member (e.g., hold the member in position within the chamber by contacting the member to the chamber wall or proximate to the chamber wall).

23. The apparatus of aspect 22, wherein when the equilibration member is magnetically engaged it is positioned proximate to, but not in direct contact with, the lower portion of the chamber wall (e.g., when the chamber is a VRC the equilibration member is held against a support such as the annular element of aspect 21 which is in contact with the substantially planer lower surface of the cylinder).

24. The apparatus of any one of aspects 22 or 23, wherein the magnet or magnetically susceptible material is positioned on or in the wall of the chamber where the central axis of the chamber passes through the wall (below the bisecting plane).

25. The apparatus of any preceding aspect, wherein the volume of the chamber is less than 2.5 times (e.g., less than 2.25, 2.0, 1.75, 1.6, 1.5, 1.4, 1.3, 1.2 or 1.1 times) the volume of the equilibration member.

26. The apparatus of any preceding aspect wherein the apparatus comprises at least one temperature sensor (e.g., two or more independent temperature sensors) that can separately monitor the temperature of: fluid entering the equilibrator via the liquid inlet 1, fluid exiting the equilibrator via liquid outlet 2, the equilibration member, and/or liquid on the equilibration member's surface.

27. The apparatus of aspect 26, wherein at least one temperature sensor is in contact with the equilibration member or wherein at least one temperature sensor is a non-contact pyrometer (one- or two-color pyrometer).

28. The apparatus of any preceding aspect, wherein the equilibration member is comprised of a material having a thermal conductivity from 10 to 200 W⁻¹K⁻¹ or more than 200 W⁻¹K⁻¹.

29. The apparatus of any preceding aspect, wherein the equilibration member is comprised of a material having a thermal conductivity from 10 to 50 W⁻¹K⁻¹ or from 50 to 100 W⁻¹K⁻¹.

30. The apparatus of any preceding aspect, wherein the equilibration member is comprised of a material having a thermal conductivity from 100 to 200 W⁻¹K⁻¹ or greater than 200 W⁻¹K.

31. The apparatus of any preceding aspect, wherein:
the liquid outlet is in fluid communication with a liquid reservoir 60 to receive the outflow of liquid (e.g., carrier liquid) from the chamber;
the liquid reservoir is in fluid communication with the liquid inlet; and
the liquid reservoir has a liquid capacity and a headspace 63 for gas (e.g., carrier gas) above the liquid in the reservoir.

32. The apparatus of aspect 31, wherein the apparatus comprises a sample injector in fluid communication with the liquid reservoir and the liquid inlet, with the sample injector positioned to receive liquid from the reservoir and to direct all or part of the liquid from the reservoir to the liquid inlet of the chamber (optionally returning part of the liquid to the reservoir to control inlet pressure and/or flow).

33. The apparatus of aspect 32, wherein: the liquid inlet, chamber, the liquid outlet, the sample injector, and the liquid reservoir are all in fluid communication and form a closed, or substantially closed, fluid path (e.g., a carrier liquid loop) through which liquid (e.g., carrier liquid and/or sample) may be circulated (e.g., recirculated).

34. The apparatus of any of aspects 31-33, wherein the liquid reservoir further comprises a headspace in gas communication with the gas inlet, or both the gas inlet and gas outlet, and wherein the reservoir's headspace is in direct or indirect gas communication with the chamber through one or both of the gas inlet and/or gas outlet.

35. The apparatus of aspect 34, wherein the gas inlet, chamber, gas outlet, and reservoir's headspace comprise a closed or substantially closed carrier gas loop for circulating (e.g., recirculating) one or more gases (e.g., a carrier gas with or without an analyte gas).

36. The apparatus of aspect 34 or 35, wherein the carrier gas loop for circulating one or more gases (e.g., carrier gas and/or a gas standard for calibration) optionally comprises a dryer/dehumidifier and/or one or more sensors 16.

37. The apparatus of any of aspects 31-33, wherein the at least one temperature control mechanism comprises a reservoir temperature control element 61 that provides heating and/or cooling of liquid 62 that may be present in(or introduced into) the reservoir 60.

38. The apparatus of any preceding aspect, wherein the at least one temperature control mechanism comprises a liquid inlet temperature control element 27 that provides heating and/or cooling to liquid entering the chamber.

39. The apparatus of any preceding aspect, wherein one or more of the at least one temperature control mechanism provides heating and/or cooling to the equilibration member, and the equilibration member optionally comprises one or more temperature sensors.

40. The apparatus of aspect 39, wherein the equilibration member is fully or partially filled with a heat-exchange fluid.

41. The apparatus of aspect 40, wherein a temperature control mechanism comprises a heat-exchange fluid circulating between the equilibration member and a heating and/or cooling unit 33.

42. The apparatus of aspect 41, wherein the heat-exchange fluid is in fluid (liquid) communication with the heating and/or cooling unit 33 and can circulate (e.g., be circulated by pumping) between the interior of the equilibration member and the heat-exchange fluid heating and/or cooling unit 33 (via heat-exchange fluid input line 31 and return line 32), wherein the apparatus optionally comprises a temperature sensor in contact with the heat-exchange fluid. See, e.g., FIG. 2D.

43. The apparatus of aspect 42, wherein the heating and/or cooling unit 33 is located external to the chamber.

44. The apparatus of any of aspects 1-43, wherein the at least one temperature control mechanism comprises a cooling unit 40, and the equilibration member is cooled by circulation of a refrigerant or cooled liquid (via coolant input line 41 and return line 42) between the cooling unit 40 and the equilibration member or a cooling element 44 therein.

45. The apparatus of aspect 44, wherein the cooling unit 40 is located external to the chamber.

46. The apparatus of any preceding aspect, wherein the at least one temperature control mechanism comprises a liquid inlet temperature control element 27 and/or a gas inlet heating and/or cooling element 28.

47. The apparatus of any preceding aspect, wherein at least one temperature control mechanism (e.g., two, three, or all temperature control mechanisms) comprises one or more electrical resistance heating elements (e.g., metal, ceramic and semiconductor composite heating elements, positive and negative temperature coefficient thick film heaters), radiant heating elements, induction heating elements, thermoelectric heating/cooling elements (e.g., Peltier elements), microwave heating elements, or a combination thereof.

48. The apparatus of aspect 47, wherein the apparatus comprises a reservoir temperature control mechanism that comprises one or more electrical resistance heating elements.

49. The apparatus of aspect 47, wherein the one or more temperature control mechanisms comprise the liquid inlet temperature control element 27 and/or the gas inlet heating and/or cooling element 28, at least one of which comprise an electrical resistance heating element, radiant heating element, or thermoelectric heating/cooling element selected independently.

50. The apparatus of any preceding aspect, wherein the at least one temperature control mechanism comprises one or more electrical resistance heating elements (e.g., metal, ceramic and semiconductor composite heating elements, positive and negative temperature coefficient thick film heaters), radiant heating elements, induction heating elements, thermoelectric heating/cooling elements (e.g., Peltier elements), microwave heating elements, or a combination thereof located inside the equilibration member. For example, the equilibration member has an inner surface and one or more of the temperature control elements is in thermal contact with the inner surface.

51. The apparatus of any preceding aspect, wherein at least one of the one or more electrical resistance heating elements, radiant heating elements, or thermoelectric heating/cooling elements is in thermal contact with the equilibration member (e.g., in thermal contact with an inner surface of the equilibration member).

52. The apparatus of any preceding aspect, further comprising a sensor for detecting one or more gases.

53. The apparatus of any preceding aspect, further comprising a sensor for carbon dioxide and/or carbon monoxide.

54. The apparatus of any preceding aspect, further comprising a sensor for hydrocarbons (e.g., a sensor to detect flammable hydrocarbon vapors in the chamber).

55. The apparatus of any of aspects 1-54, wherein (i) the liquid outlet is of an adjustable diameter to accommodate a range of liquid flow rates, and wherein liquid flowing through the outlet creates a seal that limits (e.g., substantially or completely prohibits) gas from entering or exiting the equilibrium chamber by way of the liquid outlet, (ii) the liquid outlet comprises a one way valve to permit liquid to exit the chamber, or (iii) the liquid outlet comprises a liquid trap (e.g., a p-trap). When the liquid outlet is not part of a closed or substantially closed loop for circulating liquid (e.g., carrier liquid or sample liquid) and permits water to exit the chamber and return to the environment, the water exiting the chamber via the liquid outlet can form a seal that prohibits (resists) gas exchange into or out of the chamber by way of the liquid outlet.

56. The apparatus of any of aspects 31-55, wherein the chamber is located vertically above the reservoir.

57. The apparatus of aspect 56, wherein the central axis of the chamber passes through the reservoir.

58. The apparatus of aspect 57, wherein the liquid outlet 2 is located in the wall of the chamber or in the lower surface of the chamber 45.

59. The apparatus of any of aspects 1-58 wherein the gas inlet 3 is positioned in the wall of the chamber below the bisecting plane of the equilibration member when located in the chamber and a plane that is perpendicular to the central axis and parallel to a plane passing through the liquid outlet 2 (see, e.g., FIG. 2A or 3).

60. A method of determining the amount of one or more gases and/or one or more volatile substances present in a liquid (e.g., a test liquid) for a first period of time comprising the steps:
i) providing an apparatus of any one of aspects 1-59 comprising one or more sensors for one or more independently selected analytes;
ii) introducing the liquid at an initial temperature (T1) into the chamber of the apparatus by way of the liquid inlet beginning at an initial time (t1) of a first time period such that it passes over the equilibration member thereby forming a falling film over all or part of the equilibration member's surface, and exits the apparatus by way of the liquid outlet;
iii) continuing the introduction of the liquid while optionally operating the temperature control mechanism to stabilize (maintain) the temperature of the liquid in a range that (a) encompasses the initial temperature (T1), (b) is in a range above the initial temperature, or (c) is in a range that is below the initial temperature;
iv) directing a carrier gas into the chamber of the apparatus by way of the gas inlet such that it flows over the equilibration member in a direction that is counter current or substantially counter current to the flow of the liquid, and exits the chamber of the apparatus by way of the gas outlet;
v) directing all or part of the gas that exits the chamber to one or more sensors that produce an output corresponding to the amount of at least one of the one or more gases and/or one or more volatile substances present in the liquid (e.g., the sensor(s) of an analytical instrument capable of detecting and/or measuring the amount of the one or more gases and/or one or more volatile substances); and
vi) based on the output of the one or more sensors, determining the amount of the one or more gases and/or one or more volatile substances present in the liquid during the first period of time or any period of time subsequent to the first period of time for which the one or more sensors produces output.

61. The method of aspect 60, wherein the one or more sensors receive a flow of gas exiting the chamber during the first period of time and determining the amount of the one or more gases and/or one or more volatile substances present in the liquid based on the output of the sensor is conducted concurrently with the sensor transmitting the output, or wherein the sensor output is stored (e.g., transiently or permanently) and later used for determining the amount of at least one of the one or more gases and/or one or more volatile substances.

62. The method of aspect 60, wherein directing all or part of the gas that exits the chamber to one or more sensors comprises taking samples of the gas and subsequently contacting the samples with the one or more sensors for determining the amount of the one or more gases and/or one or more volatile substances present in the liquid based on the output of the sensor.

63. The method of any of aspects 60-62, further comprising determining the amount of at least one of the one or more gases and/or one or more volatile substances present in the liquid for one or more periods of time subsequent to the first period of time (e.g., for at least a second period of time).

64. The method of any of aspects 60-63, wherein the first period of time is less than about 10 seconds or less than about 1 minute.

65. The method of any of aspects 60-63, wherein the first period of time is greater than about 1 minute and less than about 6 minutes, or greater than about 6 minutes and less than about 1 hour.

66. The method of any of aspects 60-65, wherein any one or more periods of time subsequent to the first period of time (e.g., each period of time subsequent to the first period of time) for which determining the amount of at least one of the one or more gases and/or one or more volatile substances present in the liquid is conducted is a period of time selected independently (i.e., independent of the first period of time and all other periods of time subsequent to the first period of time).

67. The method of any of aspects 64-65, wherein any one or more periods of time subsequent to the first period of time (e.g., each period of time subsequent to the first period of time) for which determining the amount of at least one of the one or more gases and/or one or more volatile substances present in the liquid is conducted is a period of time less than or equal to the first period of time.

68. The method of any of aspects 64-65, wherein any one or more periods of time subsequent to the first period of time (e.g., each period of time subsequent to the first period of time) for which determining the amount of at least one of the one or more gases and/or one or more volatile substances present in the liquid is conducted is a period of time greater than or equal to the first period of time.

69. The method of any of aspects 63-68, wherein there is an interval of time between any two or more periods of time (e.g., sequential periods of time) for which the amount of at least one of the one or more gases and/or one or more volatile substances present in the liquid is determined.

70. The method of aspect 69, wherein the interval of time between any two or more periods of time for which the amount of at least one of the one or more gases and/or one or more volatile substances present in the liquid is determined is an independently selected interval of time.

71. The method of aspect 69, wherein the interval of time between any two or more periods of time for which the amount of at least one of the one or more gases and/or one or more volatile substances is determined is a period of time equal or substantially equal to the first period of time.

72. The method of any of aspects 60-71, wherein the temperature of the liquid during all or part of the first period of time and/or during all or part of any one or more subsequent time periods (e.g., all or part of each subsequent time period) for which the amount of at least one of the one or more gases and/or one or more volatile substances present in the liquid is determined is a temperature in a range that encompasses the initial temperature (T1).

73. The method of aspect 72 wherein the temperature range is +/−20° C. or +/−10° C. of the initial temperature (T1).

74. The method of aspect 72, wherein the temperature range is +/−5° C. or +/−2.5° C. of the initial temperature (T1).

75. The method of any of aspects 60-71, wherein the temperature of the liquid during all or part of the first period of time and/or during all or part of any one or more subsequent time periods (e.g., all or part of each subsequent time period) for which the amount of at least one of the one or more gases and/or one or more volatile substances present in the liquid is determined is a temperature in a range above the initial temperature (T1).

76. The method of aspect 75, wherein the temperature range is less than 10° C. above the initial temperature (T1) or 10-20° C. above the initial temperature (T1).

77. The method of aspect 75, wherein the temperature range is 20-30° C. above the initial temperature or more than 30° C. above the initial temperature (T1).

78. The method of any of aspects 60-71, wherein the temperature of the liquid during all or part of the first period of time and/or during all or part of any one or more subsequent time periods (e.g., all or part of each subsequent time period) for which the amount of at least one of the one or more gases and/or one or more volatile substances present in the liquid is determined is a temperature in a range below the initial temperature (T1).

79. The method of aspect 78, wherein the temperature range is less than 10° C. below the initial temperature (T1) or 10-20° C. below the initial temperature (T1).

80. The method of aspect 78, wherein the temperature range is 20-30° C. below the initial temperature (T1) or more than 30° C. below the initial temperature (T1).

81. The method of any of aspects 72-80, wherein the temperature of the liquid (liquid in the equilibrator equilibrating with the carrier gas stream) is determined either on a surface of the equilibration member adjacent (e.g., proximate) to the liquid outlet 2, or alternatively, proximate to, at, or within the liquid outlet 2.

82. The method of aspect 81, wherein the temperature of the liquid is determined at or within the liquid outlet.

83. The method of any of aspects 72-80, wherein the temperature of the liquid (liquid in the equilibrator equilibrating with the carrier gas stream) is determined on an inner surface of the equilibrator adjacent (e.g., proximate) to the liquid outlet 2.

84. A method of determining the amount of one or more gases and/or one or more volatile substances present in a sample of test liquid comprising the steps:
i) providing an apparatus of any one of aspects 34-59 comprising one or more sensors for one or more independently selected analytes, wherein (a) a volume of carrier liquid is recirculated through a closed or substantially closed carrier liquid loop comprising the liquid inlet, the chamber, the liquid outlet, the sample injector, and the liquid reservoir while optionally operating the temperature control mechanism to stabilize (maintain) the temperature of the liquid in a temperature range, and (b) a volume of carrier gas is recirculated through a closed or substantially closed carrier gas loop comprising the gas inlet, the chamber, the gas outlet, and the reservoir's headspace in a direction that is counter current or substantially counter current to the flow of the carrier liquid (in the chamber), and exits the chamber of the apparatus by way of the gas outlet;
ii) introducing a sample of the test liquid (a sample of discrete known volume) into the carrier liquid by way of a sample injector, the sample of test liquid and carrier liquid entering the chamber of the apparatus by way of the liquid inlet such that it passes over the equilibration member, thereby forming a falling film over all or part of the equilibration member's surface, and exits the apparatus by way of the liquid outlet;
iii) directing all or part of the gas that exits the chamber to one or more sensors that produce an output corresponding to the amount of at least one of the one or more gases and/or one or more volatile substances present in the liquid (e.g., the sensor(s) of an analytical instrument capable of detecting and/or measuring the amount of the one or more gases and/or one or more volatile substances); and
iv) based on the output of the one or more sensors, determining the amount of the one or more gases and/or one or more volatile substances present in the sample.

85. The method of aspect 84 wherein the volume of the carrier liquid (total carrier liquid in the apparatus) is at least 10 times or at least 15 times the volume of the carrier gas. The total carrier liquid in the apparatus includes the carrier liquid in the liquid reservoir, the chamber, and any tubing (lines) connecting them.

86. The method of aspect 84, wherein the volume of the carrier liquid (total carrier liquid in the apparatus) is at least 20 times or at least 25 times the volume of the carrier gas.

87. The method of aspect 84, wherein the volume of the carrier liquid (total carrier liquid in the apparatus) is at least 30 times or at least 50 times the volume of the carrier gas.

88. The method of any of aspects 84-87, wherein the volume of the sample of test liquid is from about 0.1% to about 1% or from about 1% to about 5% of the volume of the carrier liquid.

89. The method of any of aspects 60-88, wherein the carrier gas is selected from the group consisting of air, nitrogen, an inert gas (e.g., argon, neon, xenon, or helium), hydrogen, oxygen or a mixture of any thereof.

90. The method of any one of aspects 60-89, wherein at least one of the one or more gases and/or one or more volatile substances present in the liquid is selected from the group consisting of ammonia, CO₂, CO, sulfur oxides (sulfur dioxide), nitrogen oxides (e.g., NO or NO₂), methane, ethane, hydrocarbons, halogenated hydrocarbons, chlorofluorocarbons (CFCs), hydrofluorocarbons (HFCs), perfluorocarbons (PFCs), esters, sulfur hexafluoride (SF₆), chlorine, bromine, radon, hydrogen sulfide (H₂S), HF, HCl, HBr, and Hl.

91. The method of any one of aspects 60-90, wherein the gas of interest is methane or CO₂.

92. The method of any one of aspects 60-91, wherein the liquid comprises water.

93. The method of any one of aspects 60-92, wherein the liquid is selected from the group consisting of: salt water, sea water, brackish water, tidal water, marsh water, river water, lake water, stream water, spring water, ground water, aquifer water, pore water, geyser water, volcanic water, well water, swimming pool water, aquarium water, sewer water, industrial waste water, irrigation water, run-off from agricultural sites, run-off from mines, run-off from industrial sites, drinking water treatment plant water, and sewage treatment water.

94. The method of any one of aspects 60-93, wherein the liquid (test liquid) comprises water, and wherein directing all or part of the gas that exits the chamber to one or more sensors further comprises providing a dryer/dehumidifier positioned between the gas outlet and one or more sensors, the dryer/dehumidifier receiving all or part of the gas that exits the chamber and removing all or part of the water vapor from the gas exiting the chamber to produce a dried gas stream, and the one or more sensors receiving all or part of the dried gas stream.

95. The method of any of aspects 84-94, wherein the temperature of the carrier liquid in the reservoir is maintained at a temperature within 10 degrees (±10° C.) or within 5 degrees (±5° C.) of the carrier liquid's temperature at the time the sample of test liquid is introduced into the carrier liquid.

96. The method of any of aspects 84-94, wherein the temperature of the carrier liquid in the reservoir is maintained at a temperature within 2 degrees (±2° C.) or within 1 degree (±1° C.) of the carrier liquid's temperature at the time the sample of test liquid is introduced into the carrier liquid.

97. The method of any of aspects 84-94, wherein the temperature of the carrier liquid at the liquid outlet is maintained at a temperature within 10 degrees (±10° C.) or within 5 degrees (±5° C.) of the carrier liquid's temperature at the time the sample of test liquid is introduced into the carrier liquid.

98. The method of any of aspects 84-94, wherein the temperature of the carrier liquid at the liquid outlet is maintained at a temperature within 2 degrees (±2° C.) or within 1 degree (±1° C.) of the carrier liquid's temperature at the time the sample of test liquid is introduced into the carrier liquid.

99. The method of any one of aspects 60-98 further comprising a step (e.g., following the last step or prior to the first step (e.g., step i of the subsequent measurement):
for a period of time, flowing gas (e.g., a “dry” gas substantially lacking the components of the liquid/carrier liquid) through the sensor and/or dryer/dehumidifier to remove all or part of the liquid vapor (e.g., water vapor) that may have condensed in the sensor and/or dryer/dehumidifier, or in the lines connected thereto.

100. The method of any one of aspects 60-99, further comprising a drying cycle that is optionally auto controlled, the drying cycle comprising:
optionally stopping liquid (e.g., a carrier liquid such as water) from flowing into the equilibrator,
causing carrier gas flow from a port (which can draw or vent a gas to the atmosphere (air), a carrier gas source, and/or calibrator gas source such as ports 20 or 21) through the dryer/dehumidifier 9, or sensor 16 and dryer/dehumidifier 9, toward the equilibrator (e.g., reverse flow) through a sample gas line 8 (which during forward flow brings gas from the equilibrator to the sensor system); and
exhausting the gas flowing from a port after passing through the dryer/dehumidifier, or the dryer/dehumidifier and the sensor, through a port 19 prior to reaching the equilibrator E and/or after passing through the equilibrator chamber 22. (Passing carrier gas through the parts of the system including the gas sample line and exhausting the gas once laden with vapors of condensed liquid removes condensation in the gas sample line between the equilibrator and the sensor system, thereby preventing system failure due to condensed liquid (e.g., water) entering into the sensor system.)

101. The method of any one of aspects 60-100, wherein, during the period when a carrier gas (e.g., air and/or calibrator gas) is flowing (e.g., from a port such as 20 or 21) through the sensor 16, a baseline measurement and/or calibration measurement is established.

102. The method of aspect 101, wherein the calibrator gas is air, and the flow of liquid is stopped at the liquid inlet.

103. The method of aspect 101, wherein the calibrator gas has a defined amount of methane and/or CO₂, and the flow of liquid is stopped at the liquid inlet.

104. The method of any one of aspects 60-103, further comprising:
providing a gas with a known amount of the gas of interest;
introducing said gas with a known amount of the gas of interest into the gas inlet (e.g., through port 22) and passing it through the equilibrator (and the dryer/dehumidifier if present) and at least one sensor of the one or more sensors, and then exhausting it from the all or part of the apparatus (e.g., the gas carrier loop) after passing through the at least one sensor (e.g., through port 21) (alternatively, introducing said gas with a known amount of the gas of interest into the at least one sensor (e.g., through port 19 or port 20) and exhausting it from all or part of the apparatus (e.g., the gas carrier loop) after passing through the at least one sensor (e.g., through port 21)); and
calibrating and/or confirming the calibration of the at least one sensor (e.g., the calibration of the at least one sensor and analytical instrument to which it is connected) while the gas with a known amount of the gas of interest is present in and/or flowing through the at least one sensor.

105. The method of any one of aspects 60-100, further comprising:
providing a liquid with a known amount of one or more gases or one or more volatile substances of interest,
introducing said liquid with the known amount of one or more gases or one or more volatile substances of interest into the liquid inlet; and
calibrating and/or confirming the calibration of at least one of the one or more sensors (e.g., the calibration of the detection system comprising the at least one sensor and analytical instrument) while the liquid with the known amount of the one or more gases or one or more volatile substances is flowing through the equilibrator.

### EXAMPLES

### Example 1. Comparison of Equilibrators with 20 and 25 cm Diameter Equilibration Members

Two equilibrators having spherical equilibration members placed inside VRC chambers with a single fluid inlet in the center of their removable planar upper surface and an outlet on the cylindrical surface about 1-1.5 cm from the bottom of the chamber were prepared (see, e.g., FIG. 3). The first equilibrator had a spherical equilibration member about 20.3 cm (about 8 inches) in diameter with a chamber volume of about 7.57 liters (about 2 gallons). The second equilibration member had a spherical equilibration member about 25.4 cm (about 10 inches) in diameter with a chamber volume of about 13.25 liters (about 3.5 gallons).

Using a semi-enclosed 400-liter tank, water was pumped from the bottom of the tank into the tops of the two falling film equilibrators in parallel and at similar flow rates. CO₂ concentrations in the test tank were manipulated by either spiking with pure CO₂ gas momentarily, or by continual bubbling with CO₂-stripped gas to drive CO₂ concentrations downward. Water flow rates ranged from approximately 50 to 100 gallons per minute. Water draining out of the bottom of the equilibrator was directed back into the tank and was recirculated. At any time, one or the other of the paired equilibrators was connected via a valve system to a closed loop gas train that led out of the top of the equilibrator chamber, through a dehumidifying apparatus, into a LI-COR LI-7000 infrared CO₂/H₂O gas analyzer and back into the bottom of the equilibrator. Air was used as the carrier gas and was circulated in the closed loop gas train at a rate of 1 liter per minute. Readings of pCO₂ were logged at 1-min intervals. The gas train was switched rapidly to alternately monitor the gas flow and determine how closely two equilibrators of different size agreed with one another when challenged with water of the exact same CO₂ content and to observe how quickly they responded to changes in dissolved gas (CO₂).

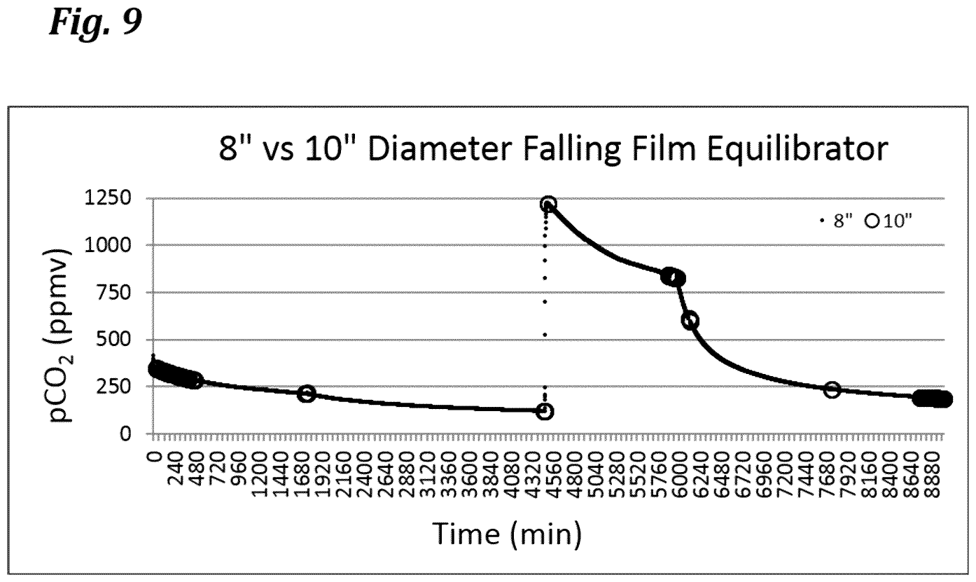

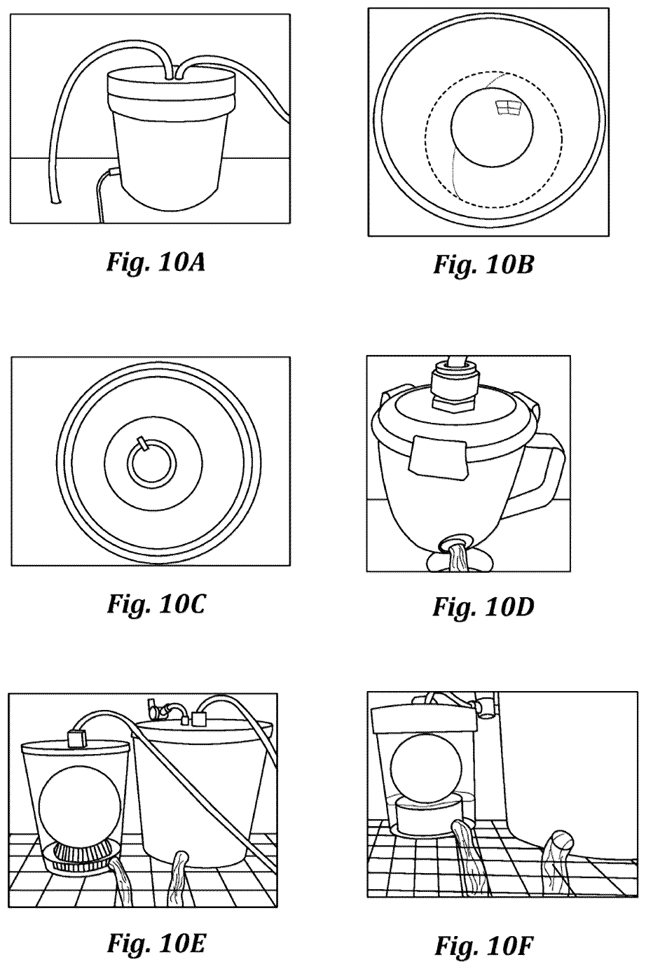

FIG. 9 shows a performance comparison of the equilibrator having an 8-inch diameter spherical equilibration member with the equilibrator having a 10-inch diameter equilibration member over a 6-day period. The 10-inch and 8-inch equilibrators were connected to the gas analyzer repeatedly and over a wide variety of CO₂ concentrations ranging from well below atmospheric concentrations to over 1200 ppmv. In all instances, the 8-inch and 10-inch equilibrators were in near exact agreement with one another.

### Example 2. Comparison of Equilibrators with 20 and 9 cm Diameter Equilibration Members

The experiment described in Example 1 was repeated using the first equilibrator from Example 1 with an equilibration member having a diameter of about 20.3 cm (about 8 inches) and a chamber volume of about 7.57 liters (about 2 gallons). For this example, the second equilibrator had a spherical equilibration member about 9.4 cm (3.7 inches) in diameter and a VRC chamber with a volume of about 1 liter (0.26 gallons). As in Example 1, the resulting measurements show a very high degree of agreement between the two equilibrators.

These and a variety of other tests of the falling film liquid-gas equilibrators described herein across broad ranges of gas (e.g., CO₂) concentrations, and liquid (e.g., water) and carrier gas (e.g., air) flow rates indicate that falling film equilibrators as described herein have the ability to produce consistent, precise, and accurate dissolved gas measurements (e.g., dissolved pCO₂ measurements) even across significantly different equilibrator dimensions. The convergence of the test results using equilibrators of different sizes suggests that complete equilibration is achieved in each case, as opposed to some arbitrary level(s) of incomplete air-water equilibration.

### Example 3. Test of Equilibrator Accuracy—Equilibrium Measurements of Water Enriched with Standard Gas CO₂/Air Mixtures

A 9 cm diameter equilibrator with a VRC chamber with a volume of about 1 liter (0.26 gallons) was attached via water- and air-tight connectors to a 5-gallon water chamber such that the system was fully closed off from the surrounding ambient atmosphere. The water chamber was enriched by bubbling the water with a certified standard CO₂/air mixture. Once the water chamber was fully enriched with the standard gas, the gas was turned off and the equilibrator was turned on. Enriched water was pumped over the equilibrator member, forming a falling film, and then drained back into the enriched water chamber. Equilibration was fully achieved after 9 minutes of run time (5 τ) and the equilibrator's headspace CO₂ concentration was measured as 7578±12.2 ppmv (mean±1 SD, n=29). This result agreed closely with the certified standard gas nominal concentration (7579 ppmv±1%), and equilibration remained stable for 30 additional minutes, until the equilibrator was turned off. This result indicates that the 9 cm diameter equilibrator equilibrates both quickly and fully (i.e., a stable equilibration with a known target standard was attained by the previously measured time constants.

### Example 4

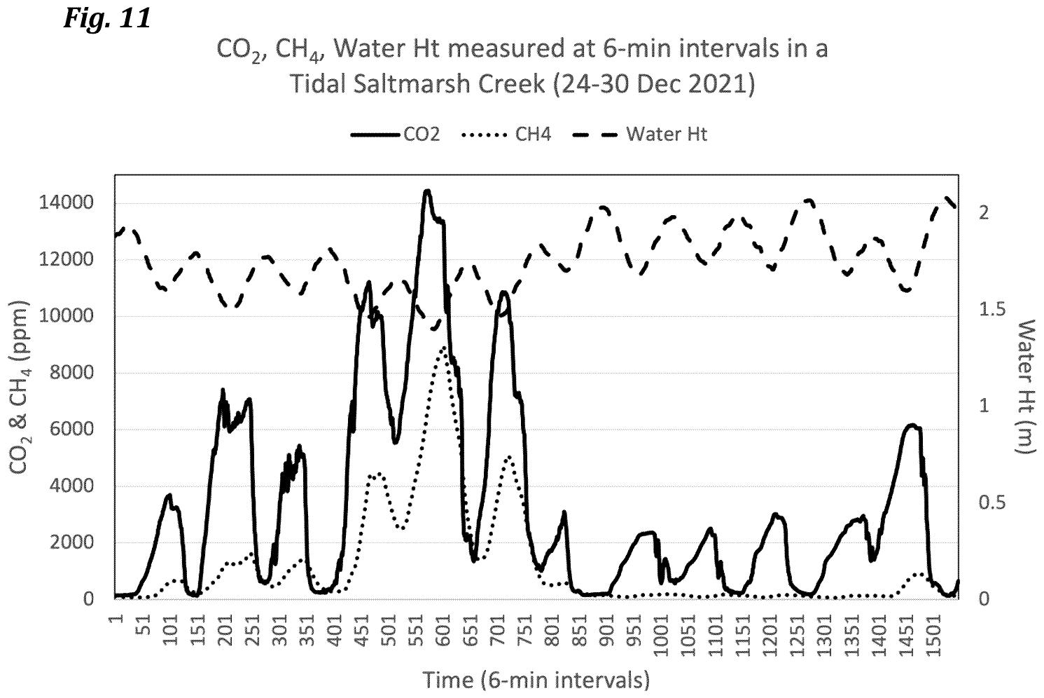

Carbon dioxide (CO₂) and methane (CH₄) in the water of a tidal salt marsh creek connected to the Chesapeake Bay in Maryland, U.S.A., and water height for the time period of December 24-30 of 2021 were measured using an equilibrator with a spherical equilibration member. Methane and carbon dioxide were measured using a Los Gatos Research (San Jose, CA, “LGR”) Ultraportable Greenhouse Gas Analyzer and are reported in parts per million (ppm). Water height (in meters) is reported relative to an arbitrary standard. CO₂ reached about 40 times atmospheric background levels, and the lowest levels of CH₄ are about 70 times atmospheric levels and reached in excess of 4,000 times atmospheric background (about 1.9 ppm). The resulting data are plotted in FIG. 11.

### Example 5

An apparatus comprising a gas/falling-liquid film equilibrator of the present disclosure configured for continuous measurement of liquid from an environmental source having a closed loop for carrier gas and continuously supplied sample from a body of water (see, e.g., FIG. 8A) is connected to a sensor for measurement of radon and at least one greenhouse gas (e.g., CO₂, CH₄, N₂O). The radon gas detector is placed in series with sensor(s) of instruments (e.g., sensor 16 of instrument(s) 17 of FIG. 8A) that measure greenhouse gases (e.g., a nondispersive infrared analyzer, cavity ringdown laser spectroscope, off-axis integrated cavity output spectroscope, photoacoustic spectroscope, etc.). In this equilibration and measurement configuration a single falling film gas-air equilibrator is used to measure more than one gas phase analyte in the carrier gas stream exiting the chamber via gas outlet 4. The analyte gases are measured simultaneously or in close succession before the carrier gas is directed back into the lower portion of the equilibrator chamber through gas inlet 3 where the carrier gas further equilibrates with the continuously supplied sample from the body of water prior to repeating its path through the carrier gas loop comprising the radon gas detector and greenhouse gas detector(s). Radon and the greenhouse gases are quantitated from the output of the sensors.

Radon is of particular interest as 1) a hydrological/chemical signature of groundwater, enabling it to be detected, quantified, and differentiated from surrounding lake, stream/river, estuarine, and/or oceanic water, where groundwater is seeping or otherwise entering these other water bodies; and 2) a contaminant of drinking water, commonly found in private drinking water wells. The described configuration and other configurations described herein allow the simultaneous measurement of radon and other trace gases or volatiles in aquifers and or drinking water wells.

1. An apparatus for contacting a gas and a liquid so as to exchange one or more gases between the gas and the liquid comprising:
a chamber comprising an outer wall that is disposed substantially symmetrically about a central axis, the outer wall defining an interior surface of the chamber, an exterior surface of the chamber, and space within the chamber;
an equilibration member within the chamber having an equilibration member outer surface, an axis of rotation, and a bisecting plane perpendicular to the axis of rotation positioned at the midpoint of the equilibration member's axis of rotation;
the equilibration member being positioned within the chamber such that its axis of rotation and the central axis of the chamber coincide or substantially coincide;
the chamber, the exterior surface of the chamber, the interior surface of the chamber, the equilibration member and its outer surface, and the space within the chamber being divided into an upper portion above the bisecting plane and a lower portion below the bisecting plane;
the space within the upper portion of the chamber being in liquid (fluid) and gas communication with the space within the lower portion of the chamber via one or more gaps between the equilibration member and the interior surface of the chamber;
at least one (i) temperature control mechanism providing heating and/or cooling of gas or liquid introduced into the apparatus and/or (ii) sample injector;
a liquid inlet located in the upper portion of the chamber positioned such that a liquid introduced into the chamber through the liquid inlet contacts the upper portion of the equilibration member outer surface;
a liquid outlet located in the lower portion of the chamber positioned such that some or all of the liquid introduced into the chamber that collects in the lower portion of the chamber (e.g., by gravity) may exit the chamber as outflow;
a gas inlet located in the wall of the lower portion of the chamber; and
a gas outlet located in the wall of the upper portion of the chamber;
wherein at least a section of the upper portion of the chamber wall is removably-resealable to the remainder of the upper portion and/or the outer wall.

2. The apparatus of claim 1 comprising at least one temperature control mechanism.

3. The apparatus of claim 2, wherein the temperature control mechanism comprises a liquid inlet temperature control element and/or at least one temperature control mechanism that provides heating and/or cooling to the equilibration member, and the equilibration member optionally comprises one or more temperature sensors.

4. The apparatus of claim 1, comprising a sample injector.

5. The apparatus of claim 4, further comprising a liquid reservoir and a closed or substantially closed carrier liquid loop that comprises the liquid inlet, chamber, liquid outlet, sample injector, and liquid reservoir, wherein a volume of carrier liquid may be circulated through the carrier liquid loop and the liquid reservoir has a headspace for a carrier gas.

6. The apparatus of claim 5, further comprising one or more temperature control mechanisms selected from the group consisting of: a liquid inlet temperature control element; at least one temperature control mechanism that provides heating and/or cooling to the equilibration member; and a reservoir temperature control element.

7. The apparatus of claim 1, wherein one or more of the chamber, equilibration member, liquid inlet, liquid outlet, gas inlet, and/or gas outlet are comprised of independently selected plastics and/or metals.

8. The apparatus of claim 7, wherein: one or more of the chamber, equilibration member, liquid inlet, liquid outlet, gas inlet, and/or gas outlet are comprised of a stainless steel.

9. The apparatus of claim 6, wherein the apparatus comprises a closed or substantially closed carrier gas loop comprising the gas inlet, the chamber, the gas outlet, and the headspace of the liquid reservoir; and
wherein the carrier gas loop optionally comprises one or more sensors for one or more analytes and/or one or more components of a dryer/dehumidifier.

10. A method of determining the amount of one or more gases and/or one or more volatile substances present in a sample of test liquid comprising the steps:
i) providing an apparatus of claim 9 comprising one or more sensors for one or more independently selected analytes wherein (a) a volume of carrier liquid is circulated through the carrier liquid loop while optionally operating the temperature control mechanism to stabilize (maintain) the temperature of the liquid in a temperature range, and (b) a carrier gas is circulated through the carrier gas loop in a direction that is counter current or substantially counter current to the flow of the carrier liquid in the chamber, and exits the chamber of the apparatus by way of the gas outlet;
ii) introducing a sample of the test liquid having a volume into the carrier liquid by way of a sample injector, the sample of test liquid and carrier liquid entering the chamber of the apparatus by way of the liquid inlet such that they pass over the equilibration member, thereby forming a falling film over all or part of the equilibration member's surface, and exit the apparatus by way of the liquid outlet;
iii) directing all or part of the gas that exits the chamber to one or more sensors that produce an output corresponding to the amount of at least one of the one or more gases and/or one or more volatile substances present in the liquid (e.g., the sensor(s) of an analytical instrument capable of detecting and/or measuring the amount of the one or more gases and/or one or more volatile substances); and
iv) based on the output of the one or more sensors, determining the amount of the one or more gases and/or one or more volatile substances present in the sample.

11. The method of claim 10, wherein the temperature of the carrier liquid in the liquid reservoir is maintained at a temperature within 10 degrees (±10° C.) or within 5 degrees (±5° C.) of the carrier liquid's temperature at the time the sample of test liquid is introduced into the carrier liquid.

12. The method of claim 10, wherein the temperature of the carrier liquid at the liquid outlet is maintained at a temperature within 10 degrees (±10° C.) or within 5 degrees (±5° C.) of the carrier liquid's temperature at the time the sample of test liquid is introduced into the carrier liquid.

13. The method of claim 10, wherein a volume of carrier liquid is removed from the carrier liquid loop equal to the volume of the sample.

14. The method of claim 10, wherein two or more analytes are measured in the carrier gas.

15. The method of claim 14, wherein the analytes are measured using a single sensor or by using one or more sensors located in series in the carrier gas loop.

16. The method of claim 14, wherein the analytes are selected from volatile organic and inorganic substances.

17. The method of claim 16, wherein the analytes are selected from CO₂, CH₄, N₂O, H₂S and radon.

18. The method of claim 10, wherein at least one analyte is measured by withdrawing a sample of carrier gas from the carrier gas loop and measuring the analyte present in an instrument that is separate from the apparatus.
